SEQUENCE LISTING (113 sequences)

SEQ ID NO: 1 - protein, 5 aa, M13 bacteriophage
IKVAV

SEQ ID NO: 2 - protein, 4 aa, M13 bacteriophage
DGEA

SEQ ID NO: 3 - protein, 4 aa, M13 bacteriophage
GRGD

SEQ ID NO: 4 - protein, 5 aa, M13 bacteriophage
YIGSR

SEQ ID NO: 5 - protein, 8 aa, M13 bacteriophage
PPKKKRKV

SEQ ID NO: 6 - protein, 29 aa, M13 bacteriophage
MMSFVSLLLVGILFWATEAEQLTKCEVFQ

SEQ ID NO: 7 - protein, 4 aa, M13 bacteriophage
KDEL

SEQ ID NO: 8 - protein, 25 aa, M13 bacteriophage
MLSLRQSIRFFKPATRTLCSSRYLL

SEQ ID NO: 9 - protein, 9 aa, M13 bacteriophage
  MISC_FEATURE (3)..(7): Xaa is any amino acid
RLXXXXXHL

SEQ ID NO: 10 - protein, 14 aa, M13 bacteriophage
DVDVPDGRGDLAYG

SEQ ID NO: 11 - protein, 15 aa, M13 bacteriophage
CGGNGEPRGDTYRAY

SEQ ID NO: 12 - protein, 7 aa, M13 bacteriophage
FHRRIKA

SEQ ID NO: 13 - protein, 14 aa, M13 bacteriophage
TMKIIPFNRLTIGG

SEQ ID NO: 14 - protein, 6 aa, M13 bacteriophage
KQAGDV

SEQ ID NO: 15 - protein, 5 aa, M13 bacteriophage
PHSRN

SEQ ID NO: 16 - protein, 4 aa, M13 bacteriophage
REDV

SEQ ID NO: 17 - protein, 5 aa, M13 bacteriophage
GFGER

SEQ ID NO: 18 - protein, 10 aa, M13 bacteriophage
RNIAEIIKDI

SEQ ID NO: 19 - protein, 4 aa, M13 bacteriophage
VAPG

SEQ ID NO: 20 - protein, 10 aa, M13 bacteriophage
KHIFSDDSSE

SEQ ID NO: 21 - protein, 8 aa, M13 bacteriophage
GPQGIWGQ

SEQ ID NO: 22 - protein, 8 aa, M13 bacteriophage
GPQGIAGQ

SEQ ID NO: 23 - protein, 7 aa, M13 bacteriophage
QPQGLAK

SEQ ID NO: 24 - protein, 4 aa, M13 bacteriophage
LGPA

SEQ ID NO: 25 - protein, 4 aa, M13 bacteriophage
APGL

SEQ ID NO: 26 - protein, 4 aa, M13 bacteriophage
YKNR

SEQ ID NO: 27 - protein, 6 aa, M13 bacteriophage
NNRDNT

SEQ ID NO: 28 - protein, 7 aa, M13 bacteriophage
YNRVSED

SEQ ID NO: 29 - protein, 6 aa, M13 bacteriophage
LIKMKP

SEQ ID NO: 30 - protein, 8 aa, M13 bacteriophage
AAAAAAAA

SEQ ID NO: 31 - protein, 7 aa, M13 bacteriophage
NQEQVSP

SEQ ID NO: 32 - protein, 6 aa, M13 bacteriophage
GLVPRG

SEQ ID NO: 33 - protein, 4 aa, M13 bacteriophage
  MISC_FEATURE (1)..(2): Xaa is any basic amino acid
XXXX

SEQ ID NO: 34 - protein, 6 aa, M13 bacteriophage
  MISC_FEATURE (1)..(1): Xaa is any hydrophilic amino acid
XXXXXX

SEQ ID NO: 35 - protein, 4 aa, M13 bacteriophage
KRSR

SEQ ID NO: 36 - protein, 6 aa, M13 bacteriophage
PRRARV

SEQ ID NO: 37 - protein, 12 aa, M13 bacteriophage
FAKLAARLYRKA

SEQ ID NO: 38 - protein, 7 aa, M13 bacteriophage
FHRRIKA

SEQ ID NO: 39 - protein, 6 aa, M13 bacteriophage
RHRHRK

SEQ ID NO: 40 - protein, 8 aa, M13 bacteriophage
LRKKLGKA

SEQ ID NO: 41 - protein, 13 aa, M13 bacteriophage
KHKGRDVILKKDV

SEQ ID NO: 42 - protein, 8 aa, M13 bacteriophage
YKKIIKKL

SEQ ID NO: 43 - protein, 11 aa, M13 bacteriophage
AEDSIKVAVDP

SEQ ID NO: 44 - protein, 8 aa, M13 bacteriophage
AASIKVAV

SEQ ID NO: 45 - protein, 11 aa, M13 bacteriophage
ADSGRGDTEDP

SEQ ID NO: 46 - protein, 5 aa, M13 bacteriophage
GRGDS

SEQ ID NO: 47 - protein, 6 aa, M13 bacteriophage
AEGDDP

SEQ ID NO: 48 - protein, 7 aa, M13 bacteriophage
ADGEADP

SEQ ID NO: 49 - protein, 6 aa, M13 bacteriophage
EQSEQS

SEQ ID NO: 50 - protein, 9 aa, M13 bacteriophage
AEQSEQSDP

SEQ ID NO: 51 - protein, 5 aa, M13 bacteriophage
AEGDD

SEQ ID NO: 52 - DNA, 6 bp, M13 bacteriophage
ctgcag

SEQ ID NO: 53 - protein, 6 aa, M13 bacteriophage
AEGDDP

SEQ ID NO: 54 - protein, 11 aa, M13 bacteriophage
  MISC_FEATURE (2)..(2): Xaa is any of Ala, Asp, Glu, Gly, or Val
AXXXXXXXXDP

SEQ ID NO: 55 - protein, 11 aa, M13 bacteriophage
  MISC_FEATURE (2)..(2): Xaa is any of Ala, Asp, Glu, Gly, or Val
AXXXRGDXXDP

SEQ ID NO: 56 - protein, 11 aa, M13 bacteriophage
AGWHWQGGGDP

SEQ ID NO: 57 - protein, 11 aa, M13 bacteriophage
  MISC_FEATURE (2)..(2): Xaa is any of Ala, Asp, Glu, Gly, or Val
AXWHWQXGGDP

SEQ ID NO: 58 - protein, 11 aa, M13 bacteriophage
  MISC_FEATURE (2)..(2): Xaa is any of Ala, Asp, Glu, Gly, or Val
AXWHWQXXGDP

SEQ ID NO: 59 - protein, 11 aa, M13 bacteriophage
  MISC_FEATURE (2)..(2): Xaa is any of Ala, Asp, Glu, Gly, or Val
AXXWHWQXXDP

SEQ ID NO: 60 - protein, 11 aa, M13 bacteriophage
  MISC_FEATURE (2)..(2): Xaa is any of Ala, Asp, Glu, Gly, or Val
AXWHWQXXXDP

SEQ ID NO: 61 - protein, 11 aa, M13 bacteriophage
  MISC_FEATURE (2)..(2): Xaa is any of Ala, Asp, Glu, Gly, or Val
AXXXEXXXXDP

SEQ ID NO: 62 - protein, 11 aa, M13 bacteriophage
  MISC_FEATURE (2)..(2): Xaa is any of Ala, Asp, Glu, Gly, or Val
AXXXEEXXXDP

SEQ ID NO: 63 - protein, 11 aa, M13 bacteriophage
  MISC_FEATURE (2)..(2): Xaa is any of Ala, Asp, Glu, Gly, or Val
AXXEEEXXXDP

SEQ ID NO: 64 - protein, 11 aa, M13 bacteriophage
  MISC_FEATURE (2)..(2): Xaa is any of Ala, Asp, Glu, Gly, or Val
AXXEEEEXXDP

SEQ ID NO: 65 - protein, 11 aa, M13 bacteriophage
  MISC_FEATURE (2)..(2): Xaa is any of Ala, Asp, Glu, Gly, or Val
AXEEEEEXXDP

SEQ ID NO: 66 - protein, 11 aa, M13 bacteriophage
  MISC_FEATURE (2)..(2): Xaa is any of Ala, Asp, Glu, Gly, or Val
AXEEEEEEXDP

SEQ ID NO: 67 - DNA, 51 bp, M13 bacteriophage
  misc_feature (13)..(13): N is A/C/G/T
atatatctgcagnknnknnknnknnknnknnknnkgatcccgcaaaagcgg

SEQ ID NO: 68 - DNA, 29 bp, M13 bacteriophage
  misc_feature (7)..(7): N is A/C/G/T
ctgcagnknnknnkcgtggtgacnnknnk

SEQ ID NO: 69 - DNA, 29 bp, M13 bacteriophage
ctgcaggctggcattggcagggcggcggc

SEQ ID NO: 70 - DNA, 29 bp, M13 bacteriophage
  misc_feature (7)..(7): N is A/C/G/T
ctgcagnktggcattggcagnnkggcggc

SEQ ID NO: 71 - DNA, 29 bp, M13 bacteriophage
  misc_feature (7)..(7): N is A/C/G/T
ctgcagnktggcattggcagnnknnkggc

SEQ ID NO: 72 - DNA, 29 bp, M13 bacteriophage
  misc_feature (7)..(7): N is A/C/G/T
ctgcagnknnktggcattggcagnnknnk

SEQ ID NO: 73 - DNA, 29 bp, M13 bacteriophage
  misc_feature (7)..(7): N is A/C/G/T
ctgcagnktggcattggcagnnknnknnk

SEQ ID NO: 74 - DNA, 29 bp, M13 bacteriophage
  misc_feature (7)..(7): N is A/C/G/T
ctgcagnknnknnkgaannknnknnknnk

SEQ ID NO: 75 - DNA, 29 bp, M13 bacteriophage
  misc_feature (7)..(7): N is A/C/G/T
ctgcagnknnknnkgaagagnnknnknnk

SEQ ID NO: 76 - DNA, 29 bp, M13 bacteriophage
  misc_feature (7)..(7): N is A/C/G/T
ctgcagnknnkgaagaggaannknnknnk

SEQ ID NO: 77 - DNA, 29 bp, M13 bacteriophage
  misc_feature (7)..(7): N is A/C/G/T
ctgcagnknnkgaagaggaagagnnknnk

SEQ ID NO: 78 - DNA, 29 bp, M13 bacteriophage
  misc_feature (7)..(7): N is A/C/G/T
ctgcagnkgaagaggaagaggaannknnk

SEQ ID NO: 79 - DNA, 29 bp, M13 bacteriophage
  misc_feature (7)..(7): N is A/C/G/T
ctgcagnkgaagaggaagaggaagagnnk

SEQ ID NO: 80 - protein, 11 aa, M13 bacteriophage
ADSGRGDTEDP

SEQ ID NO: 81 - protein, 11 aa, M13 bacteriophage
ADSGRGETEDP

SEQ ID NO: 82 - protein, 11 aa, M13 bacteriophage
ADSGGRTDEDP

SEQ ID NO: 83 - protein, 11 aa, M13 bacteriophage
AEDSIKVAVDP

SEQ ID NO: 84 - protein, 11 aa, M13 bacteriophage
AEDSIQVAVDP

SEQ ID NO: 85 - protein, 7 aa, M13 bacteriophage
ADGEADP

SEQ ID NO: 86 - protein, 11 aa, M13 bacteriophage
AEFSHPQNTDP

SEQ ID NO: 87 - protein, 11 aa, M13 bacteriophage
AEFSHPQNTDP

SEQ ID NO: 88 - protein, 11 aa, M13 bacteriophage
AEFSHPQNTDP

SEQ ID NO: 89 - protein, 11 aa, M13 bacteriophage
ADSGRGDTEDP

SEQ ID NO: 90 - protein, 11 aa, M13 bacteriophage
ADSGRGETEDP

SEQ ID NO: 91 - protein, 11 aa, M13 bacteriophage
  MISC_FEATURE (2)..(2): Xaa is any amino acid
AXWHWQXXXDP

SEQ ID NO: 92 - protein, 11 aa, M13 bacteriophage
  MISC_FEATURE (2)..(3): Xaa is any amino acid
AXXWHWQXXDP

SEQ ID NO: 93 - protein, 11 aa, M13 bacteriophage
  MISC_FEATURE (2)..(3): Xaa is any amino acid
AXXWHWQXGDP

SEQ ID NO: 94 - protein, 11 aa, M13 bacteriophage
  MISC_FEATURE (2)..(4): Xaa is any amino acid
AXXXHXXXXDP

SEQ ID NO: 95 - protein, 11 aa, M13 bacteriophage
  MISC_FEATURE (2)..(4): Xaa is any amino acid
AXXXHHXXXDP

SEQ ID NO: 96 - protein, 11 aa, M13 bacteriophage
  MISC_FEATURE (2)..(3): Xaa is any amino acid
AXXHHHXXXDP

SEQ ID NO: 97 - protein, 11 aa, M13 bacteriophage
  MISC_FEATURE (2)..(4): Xaa is any amino acid
AXXXKXXXXDP

SEQ ID NO: 98 - protein, 11 aa, M13 bacteriophage
  MISC_FEATURE (2)..(4): Xaa is any amino acid
AXXXWXXXXDP

SEQ ID NO: 99 - protein, 11 aa, M13 bacteriophage
  MISC_FEATURE (2)..(4): Xaa is any amino acid
AXXXWWXXXDP

SEQ ID NO: 100 - protein, 11 aa, M13 bacteriophage
  MISC_FEATURE (2)..(3): Xaa is any amino acid
AXXWWWXXXDP

SEQ ID NO: 101 - protein, 11 aa, M13 bacteriophage
  MISC_FEATURE (2)..(4): Xaa is any amino acid
AXXXEXXXXDP

SEQ ID NO: 102 - protein, 11 aa, M13 bacteriophage
  MISC_FEATURE (2)..(4): Xaa is any amino acid
AXXXEEXXXDP

SEQ ID NO: 103 - protein, 11 aa, M13 bacteriophage
  MISC_FEATURE (2)..(3): Xaa is any amino acid
AXXEEEXXXDP

SEQ ID NO: 104 - protein, 11 aa, M13 bacteriophage
  MISC_FEATURE (2)..(3): Xaa is any amino acid
AXXEEEEXXDP

SEQ ID NO: 105 - protein, 4 aa, M13 bacteriophage
EEEE

SEQ ID NO: 106 - protein, 14 aa, M13 bacteriophage
ACGRGESCGGGSAE

SEQ ID NO: 107 - protein, 15 aa, M13 bacteriophage
ACHPQGPPCGGGSAE

SEQ ID NO: 108 - protein, 14 aa, M13 bacteriophage
ACGRGDSCGGGSAE

SEQ ID NO: 109 - protein, 14 aa, M13 bacteriophage
ACGRGESCGGGSAE

SEQ ID NO: 110 - protein, 15 aa, M13 bacteriophage
ACHPQGPPCGGGSAE

SEQ ID NO: 111 - protein, 15 aa, M13 bacteriophage
ACHPQGPPCGGGSAE

SEQ ID NO: 112 - protein, 7 aa, M13 bacteriophage
FSHPQNT

SEQ ID NO: 113 - protein, 8 aa, M13 bacteriophage
CHPQGPPC

(19) United States
(12) Patent Application Publication
(10) Pub. No.: US 2011/0311490 A1
(43) Pub. Date: Dec. 22, 2011

(54) Recombinant Bacteriophages Useful for Tissue Engineering

(75) Inventors: Seung-Wuk Lee, Walnut Creek, CA (US); Anna Merzlyak, Berkeley, CA (US)

(73) Assignee: THE REGENTS OF THE UNIVERSITY OF CALIFORNIA, Oakland, CA (US)

(21) Appl. No.: 12/891,699

(22) Filed: Sep. 27, 2010

Related U.S. Application Data
(63) Continuation-in-part of application No. PCT/US2009/038449, filed on Mar. 26, 2009.
(60) Provisional application No. 61/039,755, filed on Mar. 26, 2008.

(51) Int. Cl. A61K 35/76; C07H 21/00; C12N 7/01

(52) U.S. Cl. 424/93.6; 435/235.1; 536/23.72

(57) ABSTRACT

The invention provides for a composition comprising a genetically engineered bacteriophage capable of guiding cell growth and polarization via signaling peptides and directionally aligned structures. The invention provides for modified bacteriophage and its uses thereof. The present invention also provides for genetically engineered phage capable of guiding cell growth, migration and/or alignment, providing essential biological effects including proliferation and/or differentiation, which can be performed by expressing specific biological motifs, such as the amino acid sequences RGD, IKVAV, DGEA and HPQ, on their coat proteins, on which functional DNA, proteins and cells can be conjugated and/or fixed thereon.

### CROSS-REFERENCE TO RELATED APPLICATIONS

This application claims priority to PCT International Patent Application No. PCT/US2009/038449, filed Mar. 26, 2009, which claims priority to U.S. Provisional Patent Application Ser. No. 61/039,755, filed Mar. 26, 2008, both of which are hereby incorporated by reference.

### STATEMENT OF GOVERNMENTAL SUPPORT

The invention described and claimed herein was made in part utilizing funds supplied by the U.S. Department of Energy under Contract No. DE-AC02-05CH11231. The government has certain rights in this invention.

### FIELD OF THE INVENTION

This invention relates generally to recombinant bacteriophages for use in tissue engineering.

### BACKGROUND OF THE INVENTION

There are currently more than 2.5 million people who live with spinal cord injuries worldwide, and an estimated 130,000 new cases are reported every year. Aside from rehabilitation therapy for lower severity cord lesions, there is currently no complete cure leading to neural regeneration. Immediately after the cord is transected and axons are severed, glial astrocyte cells, differentiated at the time of injury, invade the site to restore the blood brain barrier and secrete a thick extracellular matrix of chondroitin sulfate proteoglycans that inhibits neuronal growth. This glial scar provides both a physical barrier and a molecularly inhibitive environment for axon growth. In light of the fact that even a healthy spinal cord provides an inhibitive environment for neuronal growth, the absence of spinal cord self-regeneration is understandable. However, axons of the spinal cord neurons can extend through a transected spinal cord in vivo if given a more permissive environment, and multipotent progenitor cells identified in the spinal cord can differentiate into neurons in vitro and in vivo when transplanted into a neurogenic site such as the hippocampus. Tissue engineering for spinal cord regeneration has, to date, focused on providing a permissive and stimulating environment for neuronal regeneration. High density signaling molecule displays, nanostructured materials, and aligned fiber materials have shown promising results for promoting neuronal cell specification and growth.

Tissue regeneration is one of the most exciting and challenging research fields and requires a coordinated integration of various disciplines, including chemistry, molecular biology, materials science, and medicine. In vivo, cells are organized within extracellular matrices (ECM), filamentous protein structures that surround the cells. The ECM provides cells with mechanical support, controls the directional alignment of the cells, and directs cellular behavior by displaying bioactive peptide groups for cell attachment, growth, and differentiation. A number of previous works have demonstrated that desired tissues can be regenerated by (1) directly injecting cells into a tissue site to replenish lost cells, (2) developing three-dimensional scaffolds on which cells can grow into tissues with defined shapes, or (3) a combination of these two methods. The most common approach is to fabricate tissue scaffolds using synthetic biodegradable (or resorbable) materials such as poly(L-lactic acid) and poly(glycolic acid) or natural materials such as collagen, fibrin, and alginate. These scaffolds are seeded with the desired cells, which proliferate or differentiate to form organized cellular matrices (tissues). During the formation of the desired tissues, the scaffolding materials are degraded or resorbed. Recently, biodegradable electrospun nanofibers and self-assembling peptide amphiphiles were shown to have the ability to sustain cellular activity. However, major challenges remain for both approaches. For polymeric materials, it is difficult to ensure the high density and the proper spatial arrangement of the surface-conjugated functional groups, and self-assembling peptide amphiphiles require a laborious and expensive synthesis process for the creation of individual peptide units. Moreover, highly ordered tissue engineering scaffolds cannot be created using self-assembling peptides due to insufficient control of the self-assembling processes.

Currently there lacks a tissue engineering material that can influence cell growth chemically through display of signaling or therapeutic peptide groups, and physically by providing a directionally aligned fibrous support. Current approaches to tissue scaffold fabrication utilize synthetic biodegradable (or resorbable) materials such as poly(L-lactic acid) and poly(glycolic acid) or natural materials such as collagen, fibrin, and alginate. These scaffolds are seeded with the desired cells, which proliferate or differentiate to form organized cellular matrices (tissues). During the formation of the desired tissues, the scaffolding materials are degraded or resorbed. Recently, biodegradable electrospun nanofibers and self-assembling peptide amphiphiles were shown to have the ability to sustain cellular activity. However, major challenges remain for both approaches. For polymeric materials, it is difficult to ensure the high density and the proper spatial arrangement of the surface-conjugated functional groups, and self-assembling peptide amphiphiles require a laborious and expensive synthesis process for the creation of individual peptide units. Moreover, highly ordered tissue engineering scaffolds cannot be created using self-assembling peptides due to insufficient control of the self-assembling processes.

### SUMMARY OF THE INVENTION

The invention provides for a composition comprising a genetically engineered bacteriophage capable of guiding cell growth and polarization via signaling peptides and directionally aligned structures. The invention provides for modified bacteriophage and its uses thereof The present invention also provides for genetically engineered phage capable of guiding cell growth, migration and/or alignment, providing essential biological effects including proliferation and/or differentiation, which can be performed by expressing specific biological motifs, such as the amino acid sequences RGD, IKVAV (SEQ ID NO:1), DGEA (SEQ ID NO:2) and HPQ, on their coat proteins, on which functional DNA, proteins and cells can be conjugated and/or fixed thereon.

The invention provides for a method of modifying a bacteriophage via genetic engineering and/or chemical conjugation such that the modified phage presents a signaling peptide, therapeutic molecules, or other functional entities. The invention also provides for the use of such a bacteriophage as a material building block for the construction of structures capable of directionally or chemically guiding cell behavior in two and three dimensions.

The invention provides for a recombinant M13 bacteriophage comprising one or more recombinant phage coat protein comprising a signal peptide capable of promoting or causing a desired biological effect.

The invention provides a method of modifying a M13 bacteriophage coat with a biomolecule of interest.

This invention provides a method of conjugating a biomolecule, such as a DNA or protein, or a cell, specific to the signaling peptide via biological and/or chemical complementary interactions to coat proteins.

The invention provides for a recombinant nucleic acid encoding a recombinant M13 bacteriophage genome capable of replicating the recombinant M13 bacteriophage of the present invention.

The invention provides for a phage matrix comprising a plurality of the recombinant M13 bacteriophages of the present invention that are directionally aligned to each other.

The invention provides for making a phage matrix of the present invention, comprising: providing a plurality of recombinant M13 phages of the present invention, and allowing for self-alignment or applying an external force to align the plurality of recombinant M13 phages.

The invention provides a tissue matrix comprising a phage matrix of the present invention further comprising viable cells in contact with said phage matrix.

The invention provides for making a tissue matrix of the present invention, comprising: providing a phage matrix of the present invention, adding one or more viable cells of interest to the phage matrix to form a tissue matrix, and optionally culturing the tissue matrix for a sufficient period of time such that the viable cells grow on the phage matrix.

The invention provides for a method of treating a subject in need of such treatment comprising: making a tissue matrix of the present invention comprising cells of the subject, and implanting the tissue matrix into the subject.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Schematic diagram of the development of novel tissue engineering materials using genetically engineered phage. The motif-displaying phage is used to construct the tissue matrix structures that can control and guide cell behavior.

FIG. 2. Phage-based nanomaterial synthesis and self-assembly. (A) Schematic diagram and its corresponding TEM image show the structure of M13 phage engineered to synthesize ZnS nanoparticles. TEM image shows filamentous phage conjugated with nanoparticles at the pIII minor coat proteins. (B) Schematic diagram of close-packed phage-nanoparticle basic building block to self-assemble nanoparticles in a periodically ordered pattern defined by the length of the phage. (C) Photograph of phage-based film grown using meniscus flowing force in Eppendorf tubes. The film has a long-range ordered self-aligned structure. (D) SEM image of the phage-based film that shows the periodic alignment of the nanoparticles (arrows) at the end of the filamentous phage. (Scale bar: 1 μm).

FIG. 3. Rendered image of phage expressing a high density of signaling IKVAV motifs (1.5×10¹³ epitopes/cm²) on the pVIII coat proteins.

FIG. 4. Self-aligned structures of the pVIII engineered phage-based materials. (A) Smectic liquid crystalline suspension of pVIII engineered phage contained in 50 ml bottles (From left to right, RGD-, Wild-type-, IKVAV (SEQ ID NO:1)-phage). (B) Polarized optical micrograph of smectic phase liquid crystalline suspension in 2 mm diameter capillary tube. (C) SEM image of aligned phage in smectic suspension of (B) after critical point drying. (D) Photograph of wet-spun phage-fibers (arrow) next to a US penny. (E) SEM image of phage-fibers (scale bar=50 μm) and (F) magnification of the red-dashed box area in (E) showing the linear alignment of the phage (scale bar=10 μm). (G) Photograph of liquid crystalline phage film (arrow) and its optical micrographs (H) and (I), exhibiting the band patterns of the smectic-aligned phage molecules (scale bar=40 μm and 10 μm, respectively).

FIG. 5. Cellular viability assay results. (A) CyQUANT assay results and (B) WST-1 assay results.

FIG. 6. Immunofluorescent staining of the neuronal cells with phage added media. A-B) Progenitor media: cells were stained for nestin (green); C-D) Neural differentiation media: cells were stained for β-tubulin (green). (A&C) no phage or (B&D) 10¹² particles/mL IKVAV (SEQ ID NO:1)-phage. All cells were counterstained with DAPI (blue). Scale bar=50 μm.

FIG. 7. Fluorescence images of the neuronal cells on phage tissue culture matrices. (A) Wild-type phage cast films that show the pits generated by the neural progenitor cells (B) and their composite image (C). (D) Wild-type phage, (E) IKVAV (SEQ ID NO:1)-phage and (F) RGD-phage. All the cover slips were stained with anti-fd antibody (red). (A-C) were stained with anti-nestin (green), (D-F) were anti-β-tubulin III (orange), and (D-F) were stained with DAPI (blue). All scale bars are 50 μm.

FIG. 8. Scanning electron microscopy images of the neuronal cells cultured on the surfaces of phage films after two days in differentiation media conditions. Neuronal cells cultured on the (A) RGD- and (B) IKVAV (SEQ ID NO:1)-phage films spread well and grew longer dendrites compared with those of (C) wild-type phage film. Neuronal cells on the wild-type phage tended to form more cell aggregates. (D) Positive control neuronal cells cultured on laminin and polyornithine coated substrates. (E) RGD- and (F) IKVAV (SEQ ID NO:1)-phage films commonly exhibited pits near the neuronal cells (red and white arrows in E-H). Red Arrows in (F-H) show the high magnification images (white dotted rectangular boxed area) of interfaces between the phage films and polyornithine coating. Elongated pits are speculated to be formed by dendrites (white arrows). Scale bars in (A-E): 100 μm. (A-D) were imaged with 60 degrees tilted angle and (E-H) were imaged upright.

FIG. 9. Immunostaining of neuronal cells after eight days of differentiation on the phage films. (A-D) Neuronal cells on RGD-phage films stained for (A) phage pVIII, (B) beta-tubulin III, and (C) GFAP (glial fibrillary acidic protein) (E-H) Neuronal cells on IKVAV (SEQ ID NO:1)-phage films, and (I-L) Neuronal cells on wild-type phage films stained in the same way. All scale bars are 50 μm.

FIG. 10. Neural cells growth in 3D phage tissue matrices. (A) Fabrication of 3D phage-tissue culture scaffolds in agarose. 300 μm×5 mm channels were generated in the 2% agarose by using micro-capillary tubes. The channels were filled with phage suspension with neural progenitor cells and cultured in proliferation and differentiation media conditions. (B) Typical nematic phase POM image of liquid crystalline phage matrices with neural progenitor cells. (C) Proliferation of the neural progenitor cells that showed the multiple colony formation in RGD-phage tissue matrices. (D) Differentiation of neural progenitor cells in the RGD-phage liquid crystalline matrices.

FIG. 11. Directional guidance of cell growth using orientationally aligned phage films. (A) Schematic diagram of alignment of the phage nanofiber using shear force on the meniscus of the phage suspensions. The long-axis of phage are aligned parallel to the shear direction. (B-D) Directional alignment of the NIH 3T3 fibroblast cells cultured on the 2D oriented phage film substrate. Black arrows indicate the shear direction. (E) Control films without phage.

FIG. 12. Directional alignment of fibroblast through viral nanofibers. Long axis of the fibroblasts (NIH 3T3) were guided through the aligned phage nanofibers. White arrows indicate the shear direction. Fibroblasts were stained in red and phage stained in blue.

FIG. 13 shows various two-dimensional (2-D) and three-dimensional (3-D) structures of recombinant M13 phage.

FIG. 14 shows phage-based self-templating supramolecular structure fabrication. (A) Schematic illustration of the phage helical structures, composed of 2700 α-helical protein subunits with five-fold helical symmetry and their liquid crystal (LC) formation. (B) Illustration of the phage film deposition process, depicting the competing interfacial forces (F_{Friction} and F_{Surface tension}) at the meniscus and the liquid crystalline phase transition of the phages. (C) POM image of a 1 mg/mL phage solution at the air/liquid/solid interface, showing that the liquid crystalline phase transition occurs only at the meniscus interface. Inset POM image shows magnified side view of meniscus area, exhibiting iridescent colors of the liquid crystalline phase of phages. (D) Schematic illustration of the phage self-templating transition at the meniscus.

FIG. 15 shows alternating nematic-cholesteric structures. (A) Optical microscopy images of the stripe pattern films made from phage solutions at concentrations of 0.1 mg/mL (left) and 1.0 mg/mL (right). Inset shows a laser light (632.8 nm) diffraction pattern from the phage film. (B) Schematic diagram of alternating nematic-cholesteric periodic structures. (C) AFM images showing that the director of the oriented phage bundles is parallel to the pulling direction in the groove (left) and is rotated 90° in the ridge (right). (D) Effects of phage concentration on spacing, width, and height of ridges.

FIG. 16 shows cholesteric helix ribbon structures. (A) Schematic illustration of the interfacial forces at the curves of the three-dimensional meniscus that induce helical rotations from both side edges. (B) Optical microscope image of curved helix ribbons on a gold substrate, showing long range ordered stripe patterns. SEM images of helical ribbon structures on the left (C) and right (D) sides of the substrate: The left side edge induced right-handed helicity (C) and the right edge induced left-handed helicity (D). (E) AFM image of the right-handed helical ribbon, showing cholesteric plywood morphology. (F) The grain boundary structures in the middle, where opposite-handed helix ribbons meet.

FIG. 17 shows chiral smectic O* nanofilament structures. Photographs of iridescent phage films on gold (A) and glass (B) substrates illuminated with white light. (C) Optical diffraction pattern of the transmitted light for the film shown in (B). (D) POM image of the grain boundary of the phage film shown in (A), exhibiting opposite polarized optical responses between the left and right sides of the film. (E) AFM image shows that the film possesses smectic O zig-zag morphology and is composed of stacked nanofilaments. (F) AFM image of the nanofilaments composed of smectic C bundle structures (inset: schematic illustration of smectic C bundles). (G) Laser light diffraction patterns observed from the film shown in (A), exhibiting inversion center symmetry depending on the areas illuminated. The patterns from the left (L) and right (R) sides exhibited mirror symmetry, whereas the middle (M) areas exhibited the L and R diffraction patterns joined together in a V shape. (H) Schematic diagram of the chiral smectic C nanofilament structure constituting the film. (tilt angle ( [symbol] ), azimuthal rotation (φ) and diameter (D) of phage bundle unit structure). (I, J) Proposed model of the chiral smectic O* nanofilament structures composed of (+) and (−) rotational smectic C* nanofilaments.

FIG. 18 shows phage-based optical films and soft/hard tissue regenerating materials. (A) Phage films deposited on a gold substrate at different pulling speeds exhibited a full spectrum of color patterns. The color change of the pattern depends on the angle of incident light (deviated from normal angle). (B) Reflectance spectra from the phage film of (A). From top to bottom: phage patterns deposited at 20, 30, 40, 50, 60, 70, and 80 μm/min. (C, D) Composite photographs of the phage films deposited at controlled pulling speeds (alternating between 20 and 1000 μm/min) on gold (C) and glass substrates (D). The reflection of incident light depended on the direction of illumination (grey arrows) (C). The phage film deposited on glass selectively filtered the three letters “CAL” underneath depending on the direction of the incident light (D). (E) POM image of the alternating smetic O* nanofilament phase and nematic phase of the phage fibers, exhibiting horizontal and vertical alignment of the phage microstructures, respectively. (F, G) Phase contrast images show that the hierarchically ordered phage films composed of the RGD-phage microstructures can guide preosteoblast cell (MC-3T3 E1) growth in desired directions along the phage microstructures. The cells were elongated horizontally on the slow-deposited film (F), and vertically on the fast-deposited film (G), responding to the orientation of the phage film microstructure. (H, I) AFM images show the cells extended their bodies parallel to the orientation of the film topography. (J) SEM image of the RGD- and EEEE (SEQ ID NO:105)-phage (1:1) based hard tissue composite materials mineralized using Ca²⁺ and PO₄³⁻ solutions (detailed method is provided in the supporting materials). Inset represents the zig-zag structures of the organic/inorganic nanocomposite materials. (K) Stiffness (Young's modulus) of the phage film was significantly increased (˜18 times) after biomineralization using Ca²⁺ and PO₄³⁻ solutions.

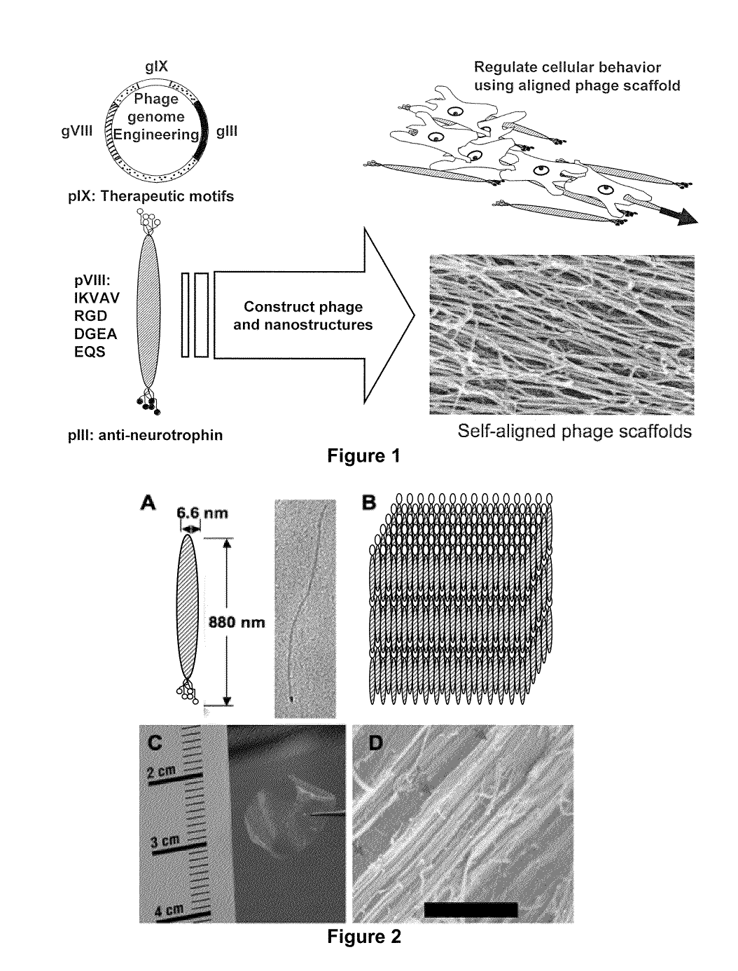

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides, and so forth.

These and other objects, advantages, and features of the invention will become apparent to those persons skilled in the art upon reading the details of the invention as more fully described below.

### Recombinant Bacteriophage

The invention allows the use of a recombinant M13 phage for tissue engineering applications. The invention allows a display of a cell signaling peptide on all 2,700 copies of major coat protein. The invention allows designed cell interaction between phage and the cell, based on engineered peptide displayed on the phage and on the structural alignment of phage matrix. The invention provides a way to display high density array of single, or multiple signaling or therapeutic peptide motifs on a directionally aligned matrix.

The invention provides for a recombinant M13 bacteriophage comprising one or more recombinant phage coat protein comprising a signal peptide capable of promoting or causing a desired biological effect, such as promoting growth of a eukaryotic cell. The recombinant phage coat protein is a recombinant pIIII, pIX, or pVIII. The signal peptide is a peptide of any suitable length that does not interfere with the self-assembly of the M13 phage. The desired biological effect includes, but is not limited to, transport, promote cell interaction, and regulate a cellular behavior, such as promoting growth of a eukaryotic cell. The signal peptide can also be any adhesion peptide sequence, enzymatic substrate peptide sequence, or heparin binding peptide sequence.

The recombinant phages are useful in having one or more the following properties: (1) natural monodisperse nanofibers which can self-assemble nanostructures, (2) high density signaling peptides or motifs can be engineered onto the major coat protein (1.5×10¹³ motif/cm²), (3) have multi-functionality on pVIII, pIII and pIX, (4) replication through bacterial host cells (such as E. coli), (5) response for external forces, (6) can identification of unknown functional motifs through phage display, and (7) ease removal from the body through lysosomal degradation.

The recombinant phages can be made in by the methods taught herein (e.g. Examples 1 and 7), with the appropriate peptide sequence as selected by one skilled in the art.

The invention provides for a genetically engineered M13 bacteriophage can be utilized to construct a novel tissue engineering material that is able to both support and influence cell growth. High density signaling molecule displays, nanostructured materials, as well as aligned fiber materials, demonstrate the phage promotes neuronal cell specification and growth. M13 phage material combines all of these qualities by providing a high-density array of peptide-based signaling molecules and therapeutic materials, achieved through genetic engineering of its coat proteins, and a directionally ordered nanofibrous liquid crystalline structure which is made possible by its nanoscale dimensions, long-rod shape and monodispersity.

In some embodiments of the invention, the M13 phage display various peptides that promote cell interaction (such as IKVAV (SEQ ID NO:1) or RGD) on all 2,700 copies of its major coat proteins. Through viability assays it is verified that these viruses are biocompatible with neuronal cells. Via microscopy studies and immunostaining it is shown that neural progenitor cells can both proliferate and differentiate when grown on viral surfaces and that there is a preference of cell interaction with the genetically engineered over wild-type phage. Utilizing SEM and bright-field microscopy it is demonstrated that such engineered phage can self assemble into directionally organized structures, which in turn can dictate the alignment and direction of cell growth. Engineered virus-based materials can be used as promising novel substrates for neural cell growth. The modified phages can further be engineered to have greater control over cell behavior at the molecular level, regeneration of various tissues, and help in the research leading to a cure of challenging conditions such as spinal cord injuries.

We have developed radically novel tissue engineering materials to control and guide cell behavior using genetically engineered M13 bacteriophage (viruses). Filamentous M13 phage has several qualities that make them attractive candidates for use as building blocks in tissue engineering scaffolds. The M13 phage has a monodisperse, long-rod shape that enables its self-assembly into directionally ordered liquid crystalline structures. Through genetic engineering, a high-density array of peptide-based signaling molecules and therapeutic materials can simultaneously be displayed on its major and minor coat proteins. We have engineered M13 bacteriophage to display various peptides that promote cell interaction (IKVAV (SEQ ID NO:1), RGD) on all 2700 copies of major coat proteins. We have verified that these viruses are biocompatible to neuronal cells using viability assays. We have shown that neural progenitor cells can both proliferate and differentiate when grown on viral surfaces and that there is a preference of specific cell interaction with the RGD- and IKVAV (SEQ ID NO:1)-peptide engineered phages over wild-type phage. Utilizing SEM and fluorescent-immunostaining microscopy we have demonstrated that such engineered phage can self assemble into directionally organized structures, which in turn dictate the alignment and direction of cell growth in 2D and 3D tissue engineering matrices. These smart and novel engineered virus-based materials can be used as promising novel substrates for neural cell growth. The success of these radically novel materials will enable to manipulate cell behavior at the molecular level and regenerating various tissues, and possibly lead to the discovery of cures for challenging diseases such as spinal cord injuries.

The M13 phage is a bacterial virus composed of a single-stranded DNA encapsulated by various major and minor coat proteins. It has a long-rod filament shape that is approximately 880 nm long and 6.6 nm wide. Through genetic modification, short peptide signaling molecules (<8 residues) can be displayed on all 2700 copies of the pVIII major coat protein, which covers most of the phage surface (>98%).

The M13 phage have several properties that make them ideal for use as a tissue engineering material. In contrast to lytic phages (T4 and lambda phage), which break the host cell wall during phage reproduction, M13 phage is non-lytic, producing little cell debris during amplification and simplifying the amplification and purification processes. Therefore, mass amplification of the virus can be easily realized through its infection of E. coli cells, resulting in a monodisperse population of the phage. Due to their monodispersity and long-rod shape, phage have the ability to self-assemble and have been extensively studied as highly organized liquid crystalline systems. The concentration of the viral suspension, ionic strength of the solution, and externally applied force fields are used to modulate viral organization in these systems and have previously been optimized for the construction of one-, two-, and three-dimensional phage-based materials. In addition, through the insertion of random gene sequences into the phage genome, a large combinatorial library can be displayed on the phage major and minor coat proteins. It has been demonstrated that semiconductor-specific phage can be isolated from the directed evolutionary screening process of phage display. These phage can synthesize various semiconductor and metallic nanocrystals and can also be self-assembled into periodically ordered films and fibril structures (FIG. 2). Such materials and structures can be used to develop high-density data storage and information processing devices.

The large surface area of the recombinant M13 phage of the present invention and their ability to present ligands in high densities make them useful as tissue engineering materials. The number of receptors that are engaged on a progenitor cell plays a pivotal role in its differentiation. The density of chemical signaling, the availability of the stimulating ligand, and the physical signaling present (e.g., surface area available for focal adhesions on nanotextured substrates) have been shown to be critical parameters in regulating cell differentiation. Aside from being a nanofibrous filament, an engineered M13 phage has the potential for presenting a very high ligand density of ˜1.5×10¹³ epitopes/cm² (3.3 nm radius, 880 nm length, 2700 pVIII units/phage).

The invention provides for a recombinant nucleic acid encoding a recombinant M13 bacteriophage genome capable of replicating the recombinant M13 bacteriophage of the present invention. The recombinant nucleic acid can also be a replicon in a suitable microorganism, such as bacterial host cell, such as E. coli. Nucleic acid sequences and methods of maintaining and replicating such replicons are well known to one skilled in the art.

### Signal Peptide Sequences

The recombinant phages comprise a short peptide motif of amino acids that is displayed on a coat protein of M13 phage. The coat protein can be pIII, pVIII, and/or pIX. In some embodiments, the peptide is 3-8 amino acids long. One skilled in the art can create partial libraries that contained randomized framing amino acids around the sequence of interest, before could successfully display the desired sequences, that also accommodated phage requirements for replication and packaging by bacteria.

Peptide sequences that promotes cell interaction include, but are not limited to, IKVAV (SEQ ID NO:1), GRGD (SEQ ID NO:3), DGEA (SEQ ID NO:2), YIGSR (SEQ ID NO:4), and RGD. IKVAV (SEQ ID NO:1) promotes neurite growth, cell differentiation and cell migration via interaction with neuronal cell receptors (LBP 110 and nucleolina). GRGD (SEQ ID NO:3) promotes cell attachment through integrin interaction. DGEA (SEQ ID NO:2) promotes cell migration and neurite growth by B1 integrin interaction. YIGSR (SEQ ID NO:4) promotes cell attachment and migration through 67 kDa laminin receptor.

Transport can include, but is not limited to, transport to the nucleus, transport to the endoplasmic reticulum, retention to the endoplasmic reticulum, transport to the mitochondrial matrix, and transport to the peroxisome. A peptide sequence capable of transport to the nucleus is -Pro-Pro-Lys-Lys-Lys-Arg-Lys-Val- (NLS) (SEQ ID NO:5). A peptide sequence capable of transport to the endoplasmic reticulum is H₂N-Met-Met-Ser-Phe-Val-Ser-Leu-Leu-Leu-Val-Gly-Ile-Leu-Phe-Trp-Ala-Thr-Glu-Ala-Glu-Gln-Leu-Thr-Lys-Cys-Glu-Val-Phe-Gln- (SEQ ID NO:6). A peptide sequence capable of retention to the endoplasmic reticulum is -Lys-Asp-Glu-Leu-COOH (SEQ ID NO:7). A peptide sequence capable of transport to the mitochondrial matrix is H₂N-Met-Leu-Ser-Leu-Arg-Gln-Ser-Ile-Arg-Phe-Phe-Lys-Pro-Ala-Thr-Arg-Thr-Leu-Cys-Ser-Ser-Arg-Tyr-Leu-Leu- (SEQ ID NO:8). Peptide sequences capable of transport to the peroxisome include, but are not limited to, -Ser-Lys-Leu-COOH (PTS1) and H₂N-Arg-Leu-X5-His-Leu- (PTS2) (SEQ ID NO:9).

Useful adhesion peptide sequences include: RGD, DVDVPDGRGDLAYG (SEQ ID NO:10), CGGNGEPRGDTYRAY (SEQ ID NO:11), FHRRIKA (SEQ ID NO:12), TMKIIPFNRLTIGG (SEQ ID NO:13), KQAGDV (SEQ ID NO:14), PHSRN (SEQ ID NO:15), REDV (SEQ ID NO:16), LDV, DGEA (SEQ ID NO:2), GFOGER (SEQ ID NO:17), IKVAV (SEQ ID NO:1), YIGSR (SEQ ID NO:5), RNIAEIIKDI (SEQ ID NO:18), VAPG (SEQ ID NO:19), KHIFSDDSSE (SEQ ID NO:20), and HAV.

Useful enzymatic substrate peptide sequences include: GPQGIWGQ (SEQ ID NO:21), GPQGIAGQ (SEQ ID NO:22), QPQGLAK (SEQ ID NO:23), LGPA (SEQ ID NO:24), APGL (SEQ ID NO:25), YKNR (SEQ ID NO:26), NNRDNT (SEQ ID NO:27), YNRVSED (SEQ ID NO:28), LIKMKP (SEQ ID NO:29), VRN, AAAAAAAA (SEQ ID NO:30), NQEQVSP (SEQ ID NO:31), and GLVPRG (SEQ ID NO:32).

Useful heparin binding peptide sequences include: BBXB (SEQ ID NO:33), XBBXBX (SEQ ID NO:34), KRSR (SEQ ID NO:35), PRRARV (SEQ ID NO:36), FAKLAARLYRKA (SEQ ID NO:37), FHRRIKA (SEQ ID NO:38), RHRHRK (SEQ ID NO:39), LRKKLGKA (SEQ ID NO:40), KHKGRDVILKKDV (SEQ ID NO:41), and YKKIIKKL (SEQ ID NO:42) (where X=any hydrophilic amino acid and B=any basic amino acid).

In some embodiments, the recombinant M13 bacteriophage of the present invention comprises a signal peptide is derived from a signal motif and/or neurotrophic factor that promotes the growth and/or maturation of neuronal cells. Such pages are useful for regenerating neural tissue. In some embodiments, the recombinant M13 bacteriophage is capable of promoting the growth and/or maturation of neuronal cells, cell adhesion, the migration of cells and/or axon extension, the formation of bundles that facilitate further extension of individual axons, or the like. Such cells include, but are not limited to, neural progenitor cells (NPCs). The NPCs can also be astrocytes and oligodendrocytes.

Neurogenesis, or the birth of functional neurons, is most active during prenatal development. During this period, the growth and maturation of neuronal cells is guided by chemically attractive and repellent signals and directional contact guidance. Signaling comes from soluble diffusible molecules, proteins bound on extracellular matrix components such as laminin, and cell adhesion molecules. Contact guidance influences both the migration of cells and axon extension. Cell migration is directed along radial glial cell extensions, and axon growth occurs along previously formed axon fiber paths, resulting in the formation of bundles that facilitate further extension of the individual axons. Neural progenitor cells (NPCs) have been identified in several areas of the adult central nervous system, including the subventricular zone (SVZ), the dentate gyms of the subgranular zone of the hippocampus, and even the spinal cord. Whereas the hippocampus and the SVZ have demonstrated neurogenesis activity, spinal cord NPCs have only been shown to generate astrocyte and oligodendrocytes in vivo. However the ability of these cells to differentiate into neurons has been demonstrated in vitro as well as in vivo by transplanting them to a more neurogenic zone of the hippocampus.

Signaling biomolecules play a critical role in controlling cellular behavior. For example, neuronal cells receive guidance as their receptors interact with extracellular matrix molecules, such as laminin, and cell adhesion molecules, such as L1 and CHL1. Various short signaling peptide motifs (RGD and IKVAV (SEQ ID NO:1)) have been shown to influence their proliferation, differentiation, migration, and axon extension. In addition, neurotrophic factors [such as nerve growth factor (NGF), brain-derived neurotrophic factor (BDNF), neurotrophins (NT-3, NT-4/5)] and their concentration gradients in extracellular environments have been shown to enhance neuronal survival, proliferation, migration, differentiation, axon growth, and synaptic plasticity.

The spinal cord contains a large number of neurons with long directionally aligned projections. Migration and neurite extension are key components in neuron differentiation and growth. Both processes are, in part, governed by contact guidance of the growth cones and the focal adhesions they make on the surrounding surfaces. Similar to the physiological alignment of axons that has been observed along other axon fibers and glial cell extensions, cell neurites have been shown to preferentially extend along the direction of the aligned texture (fibers, grooves, ridges) of their given synthetic substrates.

### Matrices of the Invention

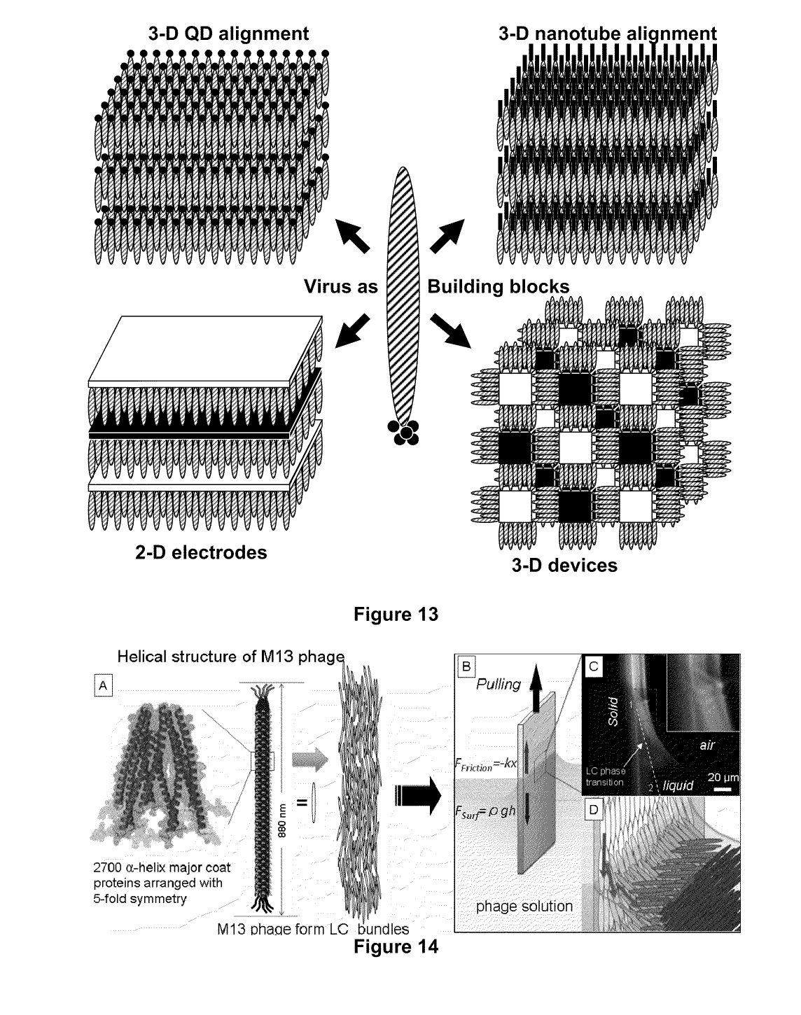

The invention provides for a phage matrix comprising a plurality of the recombinant M13 bacteriophages of the present invention are directionally aligned to each other. In some embodiments, each recombinant M13 phage is in contact with at least one or more other recombinant M13 phage. The recombinant phage of the phage matrix can be directionally aligned or oriented to each other. The phage matrix can be a 2-D phage film, or any other 2-D structure or 3-D structure (see FIG. 13).

The invention provides for making a phage matrix of the present invention, comprising: providing a plurality of recombinant M13 phages of the present invention, and allowing for self-alignment or applying an external force to align the plurality of recombinant M13 phages. The external force can be a shear force.

In some embodiments, the recombinant M13 bacteriophages of the present invention are capable of forming a self-aligned structure that promotes the directional guidance of neuronal cells in vivo.

In some embodiments, the self-aligned structure comprises an alternating nematic-cholesteric structure. The phage concentration affects spacing, width, and height of ridges within alternating nematic-cholesteric periodic structures.

In some embodiments, the self-aligned structure comprises a cholesteric helix ribbon structure. In some embodiments, the cholesteric helix ribbon structure comprises an induced left-handed helicity or an induced right-handed helicity. In some embodiments, the self-aligned structure comprises a cholesteric helix ribbon structure comprising first group of ribbons with a left-handed helicity and a second group of ribbons having a right-handed helicity, wherein there is a grain boundary structure wherein the two groups meet. In some embodiments, the self-aligned structure comprises a cholesteric plywood morphology.

In some embodiments, the self-aligned structure comprises a chiral smectic O* nanofilament structures. In some embodiments, the self-aligned structure comprises an iridescent phage films. In some embodiments, the self-aligned structure comprises a film exhibiting opposite polarized optical responses between the left and right sides of the film. In some embodiments, the self-aligned structure comprises a smectic O zig-zag morphology and is composed of stacked nanofilaments. In some embodiments, the self-aligned structure comprises of nanofilaments comprising smectic C bundle structures. In some embodiments, the self-aligned structure comprises a film exhibiting inversion center symmetry depending on the areas illuminated. In some embodiments, the film comprises patterns from the left (L) and right (R) sides which exhibited mirror symmetry, wherein the middle (M) areas exhibits the L and R diffraction patterns joined together in a V shape. In some embodiments, the self-aligned structure comprises a chiral smectic C nanofilament structure. In some embodiments, the self-aligned structure comprises chiral smectic O* nanofilament structures comprising (+) and/or (−) rotational smectic C* nanofilaments.

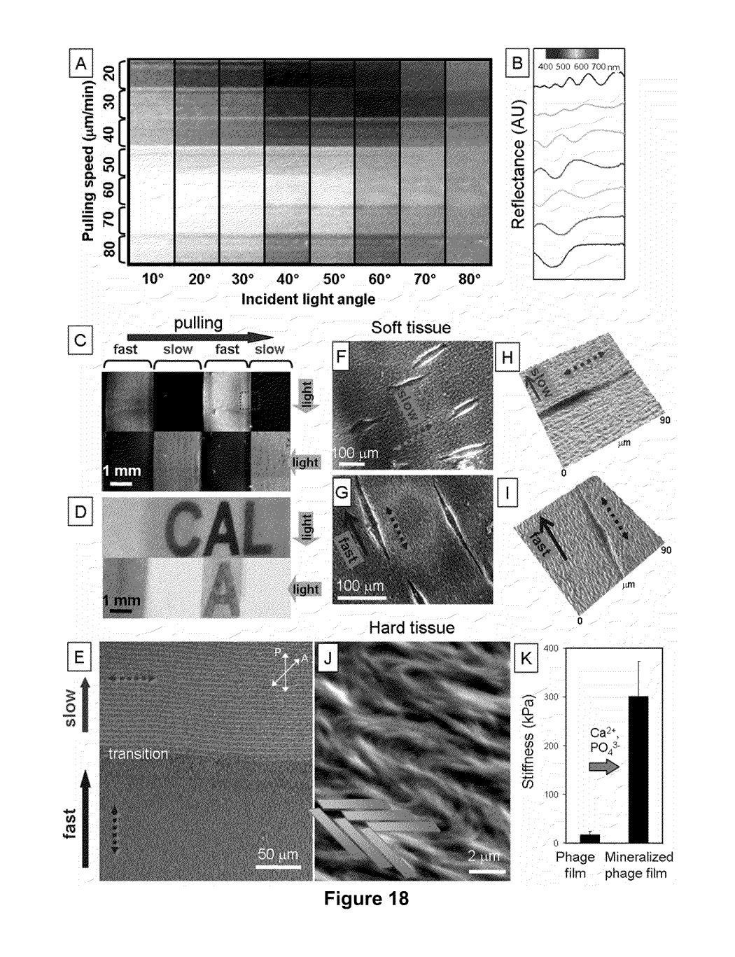

In some embodiments, the matrix comprises a self-aligned structure, such as a phage-based optical film, and a soft or hard tissue regenerating material. In some embodiments, the self-aligned structure comprises a structure shown in FIG. 18. In some embodiments, the hard tissue regenerating material is Ca²⁺ and PO₄³⁻. In some embodiments, the self-aligned structure comprises phage fibers in an alternating smetic O* nanofilament and nematic phases, exhibiting horizontal and vertical alignment of the phage fibers, respectively. In some embodiments, the matrix comprises hierarchically ordered phage films guiding the growth of cells in a desired direction along phage microstructures.

The invention provides a tissue matrix comprising a phage matrix of the present invention further comprising viable cells. The viable cells include, but are not limited to eukaryotic cells. The eukaryotic cells are animal cells, such as human cells. The viable cells can be cells of any tissue. The viable cells can be osteoblasts, chondroblasts, hepatocytes, enterocytes, urothelials, neural cells, fibroblasts. The tissue matrix may be made in vitro.

The invention provides for making a tissue matrix of the present invention, comprising: providing a phage matrix of the present invention, adding one or more of a viable cell of interest to the phage matrix to form a tissue matrix, and optionally culturing the tissue matrix for a sufficient period of time such that the viable cells grow on the phage matrix. Growing can be an increase of number cells, an increase of the cell size, and/or an adaption of the viable cell to the phage matrix, such as adhering to the phage matrix.

Removal of the Engineered Phage from the Animal Body

The recombinant M13 phage of the present invention can be removed from a body of a recipient animal by any one of two means with little side effect after the regeneration of the desired tissues. One major requirement for tissue engineering materials is that they need to be removed from the body or degraded after the regeneration of the desired tissues. Commonly used synthetic (e.g., polylactic acids and polyglycolic acids) and natural materials (e.g., collagen, fibrin, and polysaccharide materials like chitosan and glycosaminoglycans) are degradable through natural metabolic processes. Previous studies have shown that phage injected into animals are removed in two ways. The phage can be internalized through endocytosis and degraded by lysosomes. Specific ligands, such as integrin binding sequences (RGD), can increase the efficiency of internalization. Injected phage are also quickly localized to the liver and spleen and degraded by macrophages.

### Biocompatibility of Phage-Based Tissue Engineering Materials

Any non-native material in the body may cause unexpected immune responses. The biological nature of the M13 phage may also cause cytotoxicity or an immune response in surrounding cells. We expect that immune response problems encountered in the use of the engineered phage will be minor compared to those seen with conventional tissue engineering materials (polymeric and peptide amphiphile materials).

### Advantages of Phage-Based Tissue Engineering Materials Over Conventional Materials

The present invention provides for a method of constructing regenerative tissue engineering materials from genetically engineered phage has many advantages over conventional methods and materials. Such advantages are:
(1) A high density of peptide signaling motifs can easily be displayed on the pVIII major coat proteins through general molecular cloning techniques.
(2) New functional peptide sequences can be identified through directed evolutionary screening processes (phage display).
(3) The multi-functional phage can be constructed to display additional therapeutic protein motifs through modification of the pIII and pIX minor protein coats.
(4) A large quantity of identical phage building blocks can be easily prepared through amplification (non-lytic phage reproduction) using a bacterial host cell (E. coli).
(5) Phage structures can easily be self-assembled by controlling the concentration of the suspension and external force fields (i.e., magnetic, shear, or meniscus forces) due to the monodispersity and high aspect ratio of the phage.
(6) Phage are easily removed from the animal body through lysosomal degradation and removal through the liver, causing little known side-effects.
   All of these qualities could result in the fabrication of a network environment highly desirable for tissue growth and repair. Such networks could be tuned for specific cell types and activities by varying the displayed bioactive peptides and the scaffold architecture.

### Applications of the Recombinant Bacteriophages

The invention provides for a method of treating a subject in need of such treatment comprising: making a tissue matrix of the present invention comprising cells of the subject, and implanting the tissue matrix into the subject. The subject is an animal, such as a mammalian animal, such as a human. The subject can be a human patient suffering from tissue damage, such as spinal cord damage. The method can further comprise removing cells from the subject wherein the cells are the type of cells in which the subject is in need of replacing or regeneratings. The cells removed from the subject are added to a phage matrix to produce the tissue matrix. In some embodiments, the cells are neurons or neural cells, and the tissue damage is a damaged nerve or spinal cord.

The invention provides for a method of delivering a drug to a subject, comprising administering a therapeutically effective amount of a recombinant M13 phage to the subject in need of such treatment. The recombinant M13 phage comprises a signal peptide that directs the uptake or endocytosis of the phage by a particular cell and/or directs the phage to a specific part of the cell. For example, signal peptide sequences useful for this method includes those which promote transport.

The invention can be used in a medical device manufacturing industry. Additionally the platform of phage materials could be used for further academic research focused on the influence of displayed signaling and therapeutic peptide motifs on cell behavior. The engineered phage can be internalized into cells easily without any toxic effect. Therefore, the engineered phage can also be used as drug delivery vehicles.

The invention having been described, the following examples are offered to illustrate the subject invention by way of illustration, not by way of limitation.

### EXAMPLE 1

### Construction of Phage that Display Signaling Motifs

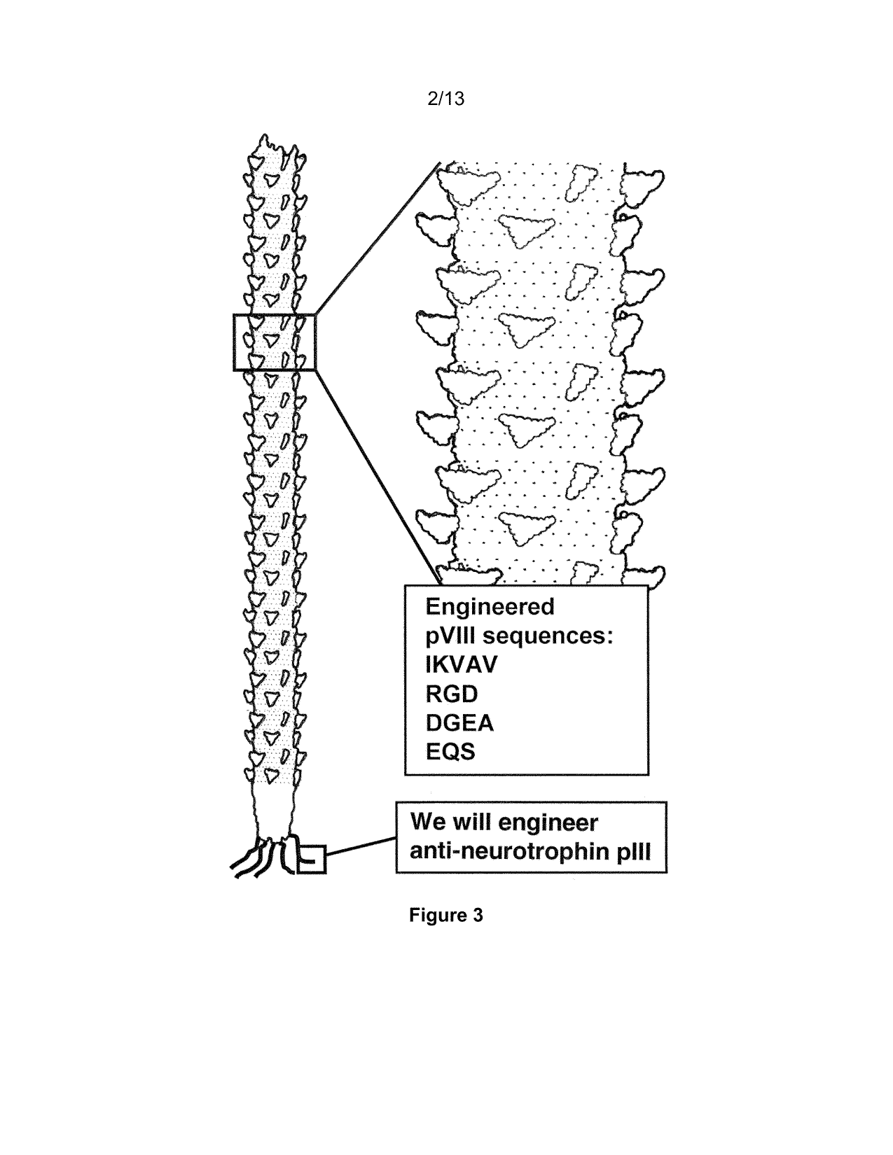

We constructed several engineered phage (FIG. 3) that displayed designated peptides at the N-termini of the pVIII major coat proteins, using methods similar to those previously reported (Nam, K. T., et al., Virus-enabled synthesis and assembly of nanowires for lithium ion battery electrodes. Science, 2006. 312(5775): p. 885-888; hereby incorporated by reference). The laminin motif IKVAV (SEQ ID NO:1) was displayed within the sequence AEDSIKVAVDP (SEQ ID NO:43) instead of the physiologically exact sequence AASIKVAV (SEQ ID NO:44). The fibronectin integrin-binding motif (RGD) was inserted within the sequence ADSGRGDTEDP (SEQ ID NO:45) instead of GRGDS (SEQ ID NO:46). The physiological sequence was initially desired but could not be successfully displayed and efficiently amplified due to disruption of the charge balances on the phage surfaces. The native pVIII molecule contains 3 negative charges within the N-terminal wild-type sequence (AEGDDP (SEQ ID NO:47)). Therefore, elimination of the negative charge inhibited the successful display of the peptide, either by reducing the infectivity of the phage (Li, Z., H. Koch, and S. Dubel, Mutations in the N-terminus of the major coat protein (pVIII, gp8) of filamentous bacteriophage affect infectivity. J Mol Microbiol Biotechnol, 2003. 6(1): p. 57-66; hereby incorporated by reference) or by disrupting the packaging and exiting mechanism of the phage particle through the bacterial membrane (Makowski, L., Structural constraints on the display of foreign peptides on filamentous bacteriophages. Gene, 1993. 128(1): p. 5-11; hereby incorporated by reference). The CHL1 motif DGEA (SEQ ID NO:2) was successfully inserted and reproduced within the sequence ADGEADP (SEQ ID NO:48). In addition, the non-biologically active motif EQSEQS (SEQ ID NO:49) was inserted in the sequence AEQSEQSDP (SEQ ID NO:50) and used as a control.

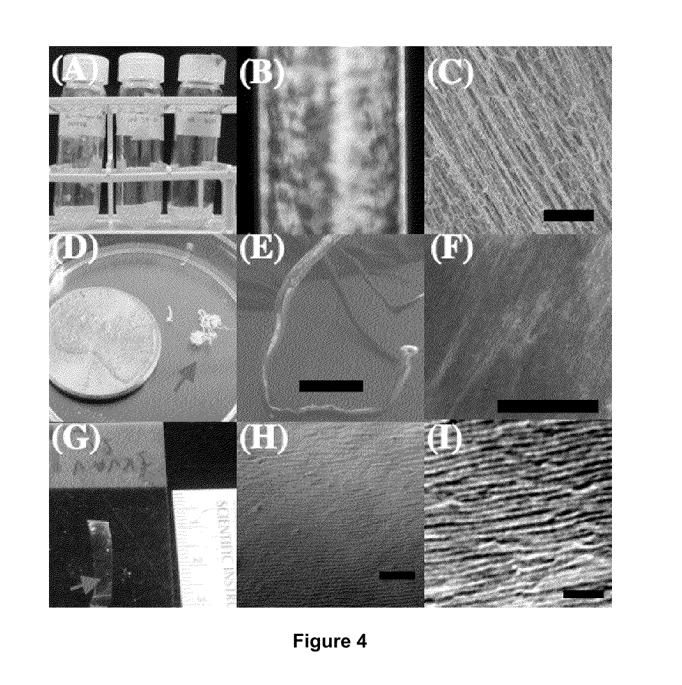

IKVAV- (SEQ ID NO:1), RGD-, and wild-type M13 phage were amplified to 12-liter volumes via E. coli infection (FIG. 4A). The phage were then purified by PEG precipitation. Prior to proceeding with cell culture work, the phage were filtered through a 0.45 μm syringe filter to further remove any remaining bacterial particles. The final concentration of amplified phage was on the order of 5×10¹³ phage/mL.

To construct and characterize phage-based tissue scaffold structures: One-dimensional phage-fibers were fabricated using electrospinning and conventional wet-spinning (Lee, S.-W. and A. M. Belcher, Virus-Based Fabrication of Micro- and Nanofibers Using Electrospinning. Nano Letters, 2004. 4(3): p. 387-390; hereby incorporated by reference). Long-range ordered smectic liquid crystalline films were fabricated through meniscus flowing force-assisted cast film processes (Lee, S. W., et al., Ordering of quantum dots using genetically engineered viruses. Science, 2002. 296(5569): p. 892-895; Lee, S.-W., S. K. Lee, and A. M. Belcher, Virus-based alignment of inorganic, organic, and biological nanosized materials. Advanced Materials (Weinheim, Germany), 2003. 15(9): p. 689-692; hereby incorporated by reference). Using these well-established techniques, we constructed one-dimensional fibers and two-dimensional cast films as described below.

Construction of liquid crystalline solutions using genetically engineered phage. We constructed liquid crystalline solutions using pVIII-engineered phage (FIG. 4A). ˜100 mg of phage pellet was suspended in 1 ml of TBS buffer to prepare a smectic phase liquid crystalline suspension. The resulting suspension was diluted to prepare the isotropic, nematic, cholesteric, and smectic phases of the liquid crystalline structures. Polarized optical micrographs were used to determine the phases [i.e., nematic (10-25 mg/ml), cholesteric (40-75 mg/ml), and smectic (90-120 mg/ml, FIG. 4B)], and the structures formed by all the engineered phage were very similar. After critical point drying the samples in the smectic phase, we visualized the individual phage nanofibers (FIG. 4C), which spontaneously aligned in the smectic suspension. The self-aligned phage-based materials were able to display the signaling motifs with directionality and, therefore, may guide the neuronal cells through direct contact guidance.

Fabrication of one-dimensional fibers. We fabricated one-dimensional fibers using mild spinning conditions by spinning the liquid crystalline suspensions (20-100 mg/ml) into an ethanol fixative (70% aqueous ethanol). FIG. 4D shows the viral fibers with IKVAV- (SEQ ID NO:1) engineered phage. Scanning electron microscopy images (FIGS. 4E and F) show that the long axes of the phage are parallel to the long axis of the viral fibers.

Fabrication of liquid crystalline films. We fabricated two-dimensional ordered liquid crystalline phage films (FIG. 4G). 1.5 mg/ml of phage suspension was cast dried on the surface of a vinyl film substrate (Thermanox™ cell culture cover slips, Nalge Nunc International, Rochester, N.Y.) in a dessicator for one week. Examination by polarized optical microscopy revealed striped band patterns in the dried sample, indicating that the phage long axes were aligned perpendicularly to the bands in a smectic liquid crystalline alignment (FIGS. 4H and I).

### EXAMPLE 2

### Effects of the Phage Materials on Neural Cell Viability and Growth

In order to investigate the cytotoxic effect of the phage materials, we tested the individual phage as additives to the cell culture media and then tested drop cast films of the wild-type phage and phage displaying IKVAV- (SEQ ID NO:1) and RGD-motifs.

Neural progenitor cell cultures: We investigated the cytotoxicity of the engineered phage on neural progenitor cells. The neural progenitor cells used in these experiments were isolated from the hippocampi of adult female Fischer 344 rats, as previously described (Saha, K., et al., Biomimetic interfacial interpenetrating polymer networks control neural stem cell behavior. J Biomed Mater Res A, 2007. 81(1): p. 240-9; hereby incorporated by reference). The cells were seeded at a density of 3x10³ cells/cm² into 96-well plates with clear bottoms (Greiner Bio-One, Monroe, N.C.) precoated with polyornithine and laminin. They were grown in 100 μL serum-free DMEM/Hams F-12 medium with N-2 supplement (Invitrogen, Carlsbad, Calif.) and 20 ng/ml bFGF (Peprotech, Rocky Hill, N.J.) at 37° C. and 5% CO₂.

Phage as a media additive: Different concentrations of constructed phage (displaying IKVAV (SEQ ID NO:1) and RGD) and wild-type phage were added to the media to approximate various potential dissolution conditions at the cell material interface. Estimating the footprint of the phage to be a 6.6×880 nm rectangle, we calculated that the cell growth area in each well (0.34 cm²⁾ could be completely covered by approximately 5×10⁹ phage. This quantity of phage was used to set a median phage concentration, and phage concentrations that were 10× greater and 10× less were tested to see if the phage produced any significant trends in cell behavior. To keep the concentration of the phage and nutrients in the media constant, 50 μL of the phage/media were changed daily.

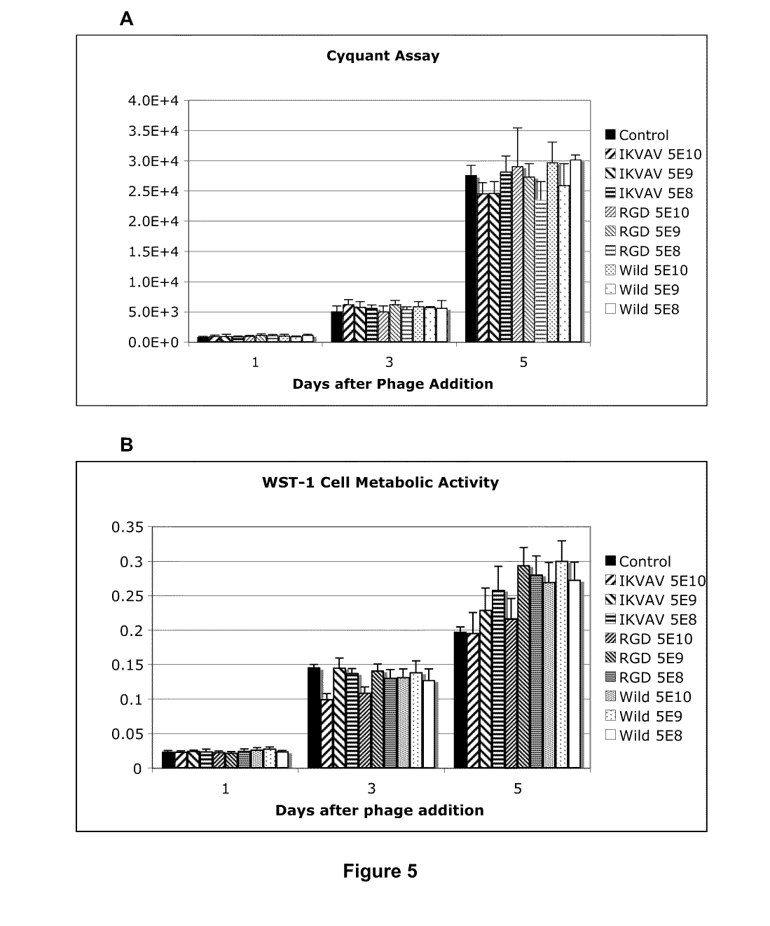

CyQUANT Assays: The proliferation and metabolic activity of the cells were measured after 1, 3, and 5 days of growth in both the presence and absence of phage. The high throughput [12] CyQUANT assay (Molecular Probes, Eugene, Oreg.) was used per manufacturer's instructions. A Safire™ microplate reader with Xflour™ Software (Tecan Systems, Inc, San Jose, Calif.) was used to take the measurements. To obtain the data, 4-6 samples were tested at each given condition. CyQUANT fluorescent dye binds to nucleic acid material in the cell, thus allowing the amount of DNA in the culture to be quantified and indicating the number of cells present at the time of the test. There was no significant difference in the proliferation rate between the control population of cells in plain media and the cells grown in the presence of phage (FIG. 5A). The doubling rate of cells is fairly steady over the duration of the experiment (19.9 hrs first 2 days, 21.4 hrs next two days). The slight slowing of cell proliferation may have resulted as the cells reached confluence (at day 5).

WST-1 Assays: We also used a WST-1 assay (Roche, Indianapolis, Ind.) to evaluate cell viability. The assay is based on the activity of mitochondrial dehydrogenase (NADH), which cleaves water soluble tetrazolium salt to formazan to produce a detectable color change. Mitochondrial activity can also be correlated to cell number and cell activity (with higher activity indicating a higher potential for proliferation and differentiation). FIG. 5B shows that the control cells in plain media exhibited approximately the same or slightly lower mitochondrial activity than the cells in the phage media. Thus, the addition of phage does not decrease the viability of the cells or their metabolic activity. As with the CyQUANT assay, we can see increases in activity with time. Whereas the increases observed in the WST-1 and CyQUANT assays correspond similarly from day 1 to day 3, the increase from day 3 to day 5 is lower for the WST-1 assay than for the CyQUANT assay.

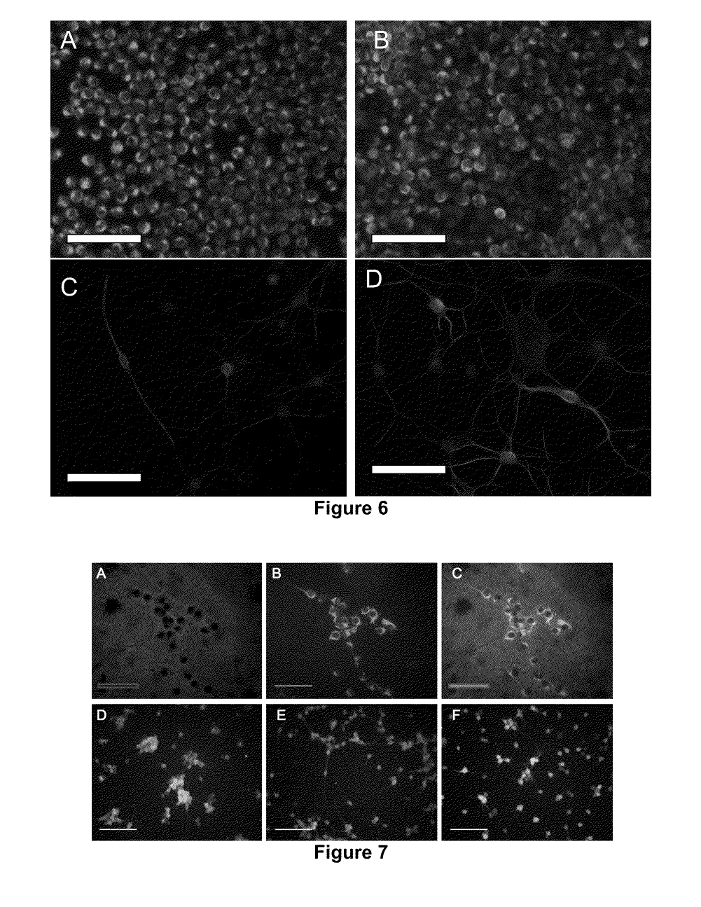

Cell Differentiation and Maintenance: To further assess the cellular responses to the phage, we performed immunostaining to visualize cell state specific markers. As in the above experiments, the cells were plated on polyornithine- and laminin-coated surfaces. The cells were grown in the presence (5×10¹⁰ phage particles) and absence of IKVAV-phage in either progenitor media supplemented with bFGF, as described above, or in neural differentiation media [serum-free DMEM/Hams F-12 medium with N-2 supplement, 1 μM retinoic Acid, and 5 μM forskolin (BioMol, Plymouth Meeting, Pa.)]. After 3-6 days in culture, the cells were fixed by formalin (Ted Pella Inc., Redding, Calif.) and their nuclei stained with DAPI (4′,6-diamidino-2-phenylindole). Mouse anti-nestin antibody (1:1000, BD Biosciences, San Jose, Calif.) was used to indicate the presence of nestin, a neural progenitor specific protein, and anti-β-tubulin III IgG was used to indicate the presence of β-tubulin III (1:400, Sigma-Aldirich), a neuron-specific neurofilament protein. FIG. 6 shows no apparent difference in expression of progenitor or neural specific proteins when the cells were grown in the presence or absence of IKVAV phage.

### EXAMPLE 3

### Effects of Phage-Films on Cellular Viability

Through immunofluorescent staining, we confirmed that neural progenitor cells can adhere to the phage-films and stay viable while growing on the substrate. In addition, the cells maintained their progenitor state and differentiated when subjected to defined media conditions. In order to prepare drop-cast phage films, 304 droplets of 10¹² phage/mL of either wild-type or genetically modified phage were deposited on vinyl cover slips (VWR) and allowed to incubate overnight at room temperature. Neural progenitor cells were seeded on the top of the phage films. After remaining in culture for 5 days, the cells were formalin-fixed, and the cover slips were double stained with anti-fd phage antibody (1:500) and anti-nestin antibody. FIGS. 7A-C shows the individual staining images from each antibody as well as a composite. These images show that the cells and phage material can be visualized concurrently. Using fluorescence imaging, we confirmed that the cells were able to adhere and differentiate into neuronal type cells on top of the phage cast film surfaces. Interestingly, we found pits generated near the areas in which the neural progenitor cells grew (FIGS. 7A-C). Such pits could have resulted from phage endocytosis by the cell or blocking of the staining due to tight binding of the cells onto the phage films. FIGS. 7D-F show that the neural progenitor cells are able to differentiate into neuronal type cells on top of the phage surfaces. The differentiated neural progenitor cells show slightly different morphologies depending on the motifs that are displayed on the phage. The differentiated cells on the IKVAV- (SEQ ID NO:1) and RGD-phage substrates show longer neurite extension (FIGS. 7E and F) than those cultured on the wild-type phage substrates (FIG. 7D). In addition, the differentiated cells adhered and spread better on the IKVAV- (SEQ ID NO:1) and RGD-phage substrates (FIGS. 7E and F) than those on the wild-type phage substrates (FIG. 7D).

### EXAMPLE 4

### Differentiation of Neuronal Progenitor Cells on 2D Phage Films

We studied the effects of the phage films displaying peptide-signaling motifs on growth and differentiation of the neural progenitor cells. We constructed the 2D films using RGD-, IKVAV- (SEQ ID NO:1), and wild-type (no insert) phages on (100) silicon substrates by casting the phage suspensions as previously reported (Lee, S. W., et al., Ordering of quantum dots using genetically engineered viruses. Science, 2002. 296(5569): p. 892-895; and Lee, S.-W., S. K. Lee, and A. M. Belcher, Virus-based alignment of inorganic, organic, and biological nanosized materials. Advanced Materials (Weinheim, Germany), 2003. 15(9): p. 689-692; hereby incorporated by reference). After the film was dried onto the silicon substrates, the film surface was rinsed two times with PBS, and neural progenitor cells were seeded on top. The cells were cultured in neural differentiation media with media changes every two days. After two and eight days of maintaining culture conditions, the silicon substrates were removed from culture media and fixed in 2% gluteraldahyde. The samples were then processed for Scanning Electron Microscopy (SEM) imaging.

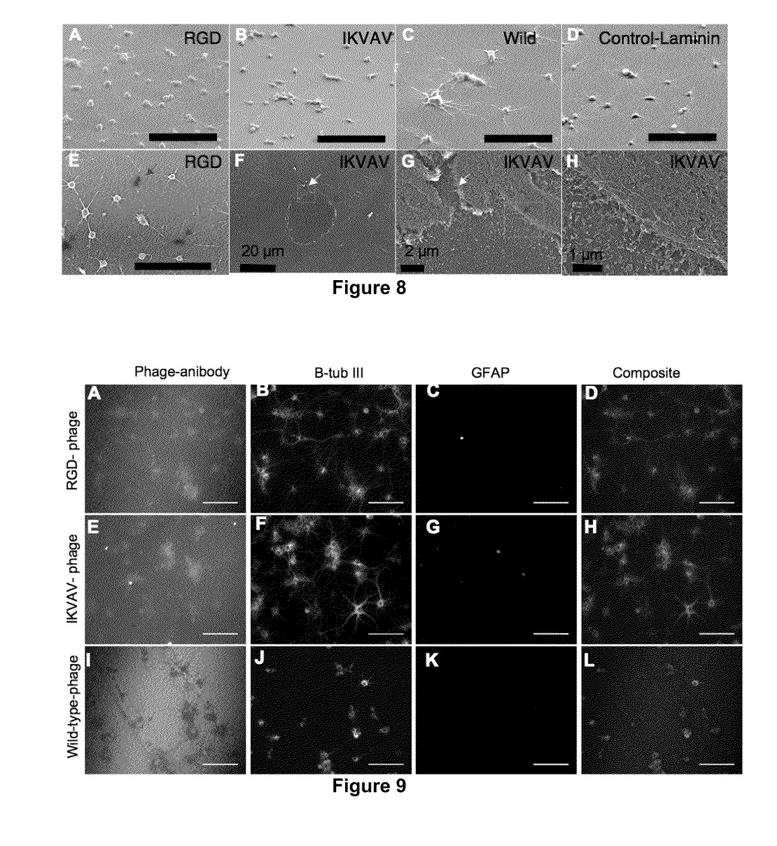

FIG. 8 shows the SEM images of differentiated neuronal cells on RGD-, IKVAV- (SEQ ID NO:1), wild-type phage films and positive control (laminin and polyornithine coated surfaces) cultured in the differentiated media conditions (see Example 3). RGD- and IKVAV- (SEQ ID NO:1) phage films (FIG. 8A-B) showed that neurons can spread on the phage film and grow long dendrites throughout the samples. Wild-type phage substrate (FIG. 8C) showed the least amount of cell spreading, the cells instead preferred to growing together in aggregates. The RGD- and IKVAV- (SEQ ID NO:1) phage films exhibited the same growing patterns of cells as observed on the positive control laminin and polyornithine coated substrates (FIG. 8D). Interestingly, we observed many pits (red arrows in FIG. 8E-H) on the phage films very similar to those of the FIG. 7A. Based on the dendrite patterns around the pits, we believed that these pits were the places that the differentiated neuronal cells have previously occupied. Potentially these pits were observed because the cells were displaced during the SEM sample preparation procedure. FIG. 8F-H shows the typical morphology of the pit at high-magnification. We believe that the neuronal cells internalized the underlying phage substrates.

We observed that the growth patterns of neuronal cells depended on the signaling peptides displayed by the phage films. FIG. 9 shows the immunostaining of the neuronal cells on the RGD-, IKVAV- (SEQ ID NO:1), and wild-type phage films after eight days of culture in the differentiation media conditions. Both individual marker staining (pVIII phage, beta-tublin III, and GFAP) as well as the composite image are presented. Neuronal cells grown on RGD- and IKVAV- (SEQ ID NO:1) phage films spread well and grew many long dendrites. Those on wild-type-phage substrates exhibited aggregated cell morphologies and much shorter dendrites than those of the cells cultured on the RGD- and IKVAV- (SEQ ID NO:1) phage films. In addition we observed greater internalization of RGD- and IKVAV- (SEQ ID NO:1) phage materials into the neuronal cells rather than wild-type phage materials. Neuronal cells stained for pVIII marker on RGD- and IKVAV- (SEQ ID NO:1) phage films exhibited much brighter fluorescence emission from neuronal cells areas, while the neuronal cells cultured on the wild-type phage films showed that the neuronal cell areas exhibited much darker emission. We rarely observed the growth of astrocytes during the culture (FIGS. 9C, G, and K).

### EXAMPLE 5

### Neuronal Cell Growth on 3D Phage Matrices

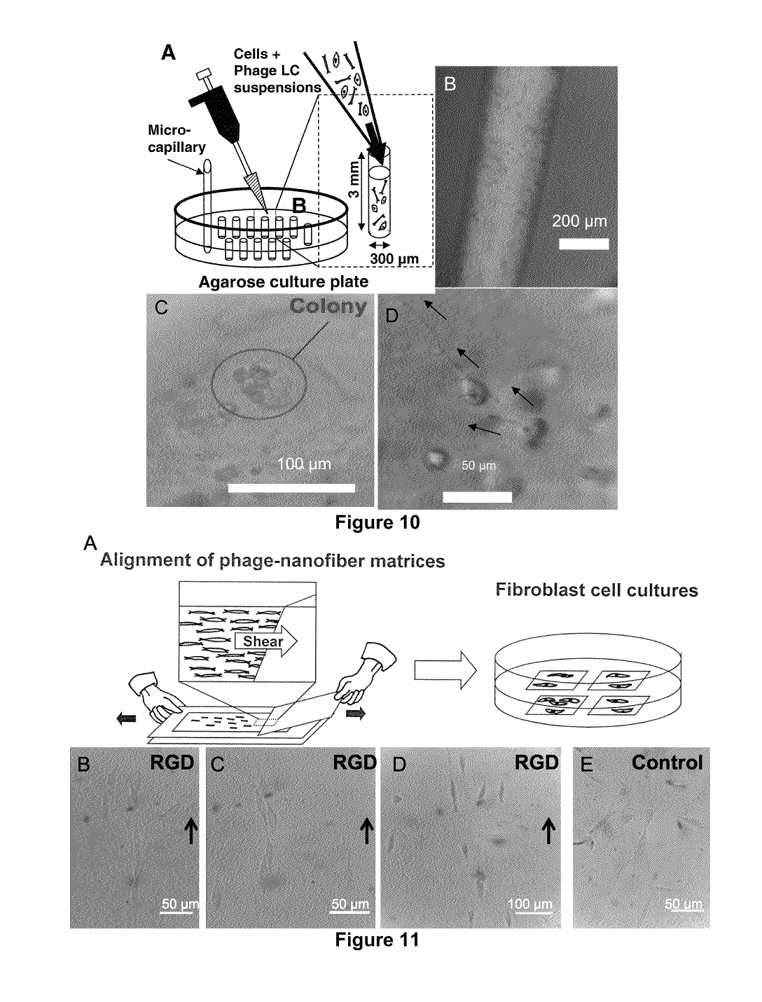

We established a method to culture neural progenitor cells within three dimensional liquid crystalline phage matrices. Additionally we have been able to successfully proliferate and differentiate the neural progenitor cells within the phage matrices. Three dimensional phage matrices were constructed using the nematic liquid crystalline suspensions of the RGD-, IKVAV- (SEQ ID NO:1) and wild-type-phages (FIG. 10A). The anisotropic properties of this material were verified by birefringence observed with polarized optical microscopy (see FIG. 10B). After preparing nematic liquid crystalline suspensions (˜10-15 mg/ml) of phage using cell growth media (DMEM/F12 media), the suspensions were homogeneously mixed with neural progenitor cells suspension (1 to 2×10⁶ cells/mL) in the same culture media. We generated channels (approximated 300 μm in diameter and 5 mm in length) within agarose filled Petri dish. The channels were then cut out and filled with nematic phage suspension containing the neural progenitor cells. After four days of culture, we observed neural progenitor cells proliferated to form multiple colonies within RGD-, IKVAV- (SEQ ID NO:1), and wild-type phage 3D matrices (FIG. 10C). Live and Dead staining verified that most of the neuronal cells were alive.

When the neural progenitor cells were grown in differentiation media conditions, the cells were able to differentiate and extend dendrites after four days of culture (FIG. 10D). The dendrite growth exhibited preferential orientation pattern grown through the direction of liquid crystalline fiber matrices (arrows in FIG. 10D). The preferential orientation of dendrites might be influenced by the orientation of phage tissue matrices.

### EXAMPLE 6

### Two-Dimensional Studies of Cell Alignment Using 3T3 Fibroblast

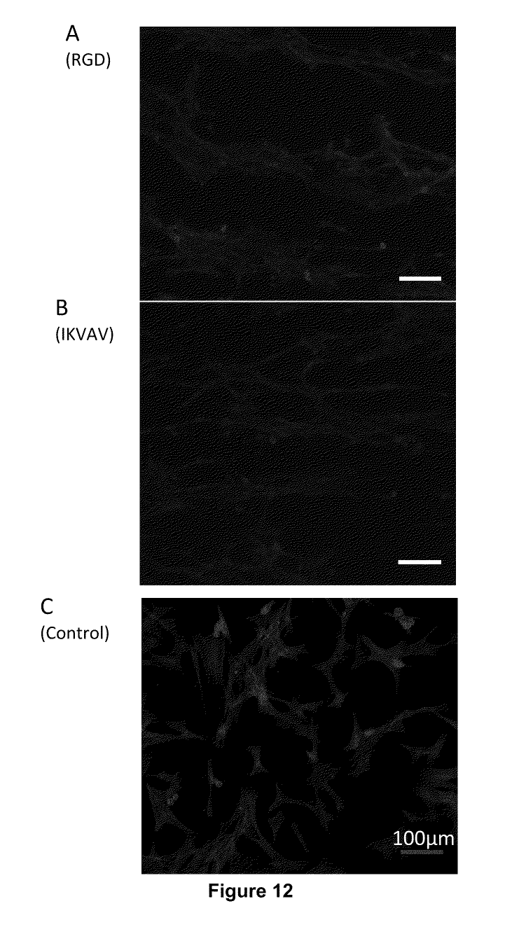

We studied the directional control of ordered phage substrate on cell polarization, by observing the growth patterns of 3T3 fibroblast cells. RGD-, IKVAV- (SEQ ID NO:1) and wild-type phage were used to construct the orientationally-ordered 2D phage film substrates using shear forces. 1 μL of the phage solution (10 mg/mL) were placed on the surface of a coverslip and sheared across the surface of the slip using another coverslip (FIG. 11A). The coverslips were then allowed to dry overnight in sterile conditions. NIH 3T3 fibroblasts were seeded on to the coverslips at a concentration of 5×10⁴ cells/cm². The cells were cultured for two days in standard conditions (DMEM media supplemented with 10% fetal bovine serum, and 1% penicillin/streptomycin at 5% CO2 and 95% humidity). The growth pattern of cells was observed with light microscopy. A coverslip with no phage was used as a control surface. FIG. 11 shows that the directional control of the fibroblast growth using genetically engineering phage tissue engineering matrices. The ordered phage surface exhibited a remarkable directional control over the polarization and alignment of the fibroblast cells. The fibroblasts were aligned though the direction that we applied the shear forces. In addition phage substrates exhibited good spreading and cell adhesion. Because the long axis of the phage are aligned parallel to the shear direction and the RGD-signaling peptides on the phage can stimulate the fibroblast cell growth, the fibroblast preferentially aligned through the direction of the long-axis of the phage nanofibers. In addition, morphologies of the NIH 3T3 fibroblast exhibited that much narrower compared with those of the control (FIG. 11E).

We successfully constructed pVIII-engineered phage that display short peptide signaling motifs (IKVAV (SEQ ID NO:1), RGD, DGEA (SEQ ID NO:2) and EQS), their control phage, and a random peptide phage library using site specific mutagenesis methods. Our studies on the effects of phage on neural progenitor cell viability using WST-1 and CyQUANT assays showed that the engineered phages have no cytotoxic effects on the neuronal cells compared to the control samples grown in the absence of phage. The differentiation experiment results showed RGD- and IKVAV- (SEQ ID NO:1) phage films showed that neurons can spread on the phage film and grow long dendrites throughout the samples. Wild-type phage substrate showed the least amount of cell spreading, the cells instead preferred to growing together in aggregates. We have demonstrated that such RGD- and IKVAV- (SEQ ID NO:1) peptide engineered phage can self assemble into directionally organized structures, which in turn dictated the alignment and direction of cell growth in 2D and 3D tissue engineering matrices. These smart and novel engineered virus-based materials can be used as novel substrates for neural cell growth and will enable to manipulate cell behavior at the molecular level and regenerating various tissues.

### EXAMPLE 7

### Construction of Phage that Display Signaling Motifs

Partial Library Method. Functional signaling peptide motifs of the nervous system extra cellular matrix (RGD and IKVAV (SEQ ID NO:1)) were expressed on pVIII of M13 phage for use as a tissue engineering matrix. The nanofilamentous M13 bacteriophage covered with 2700 copies of the major coat protein pVIII, present a great surface for a controlled dense and uniform peptide display previously shown successful in regulating cell adhesion and differentiation behaviors (K. Saha, E. F. Irwin, J. Kozhukh, D. V. Schaffer, K. E. Healy, J Biomed Mater Res A 81, 240 (2007); J. C. Schense, J. Bloch, P. Aebischer, J. A. Hubbell, Nat Biotechnol 18, 415 (2000); G. A. Silva et al., Science 303, 1352 (2004)). However, as pVIII is involved in many phage replication processes within its Escherichia coli bacteria host (D. Stopar, R. B. Spruijt, C. J. Wolfs, M. A. Hemminga, Biochim Biophys Acta 1611, 5 (2003); M. Russel, Journal of Molecular Biology 231, 689 (1993); hereby incorporated by reference), any genetic modifications or peptide additions to pVIII must be able to accommodate its biological roles in making a viable phage. To circumvent such biological censorship inherent to phage replication, we used a partially constrained insert library. Constraining a short functional motif, by itself unfavorable for expression on pVIII protein (i.e. RGD), among degenerate residues in an 8mer insert, allowed us to display these peptides on every copy of pVIII for tissue engineering. Further we constructed four chemically diverse series (His, Trp, Glu, Lys) of 8mer partial peptide libraries to systematically analyze pVIII insert expression capabilities. By constraining one to six repeated amino acids within the insert, we observed several biochemical and size related compensation mechanisms of the library residues to make the full insert favorable to expression of pVIII. This novel method, to present unfavorable peptide groups on all of the major coat proteins of the M13 bacteriophage can serve to better design and evaluate pVIII inserts and further exploit the phage as functional nanobiomaterial.

Inverse PCR method for M13 phage cloning. To present peptide motifs on every copy of M13 major coat protein an inverse PCR cloning method was adapted (G. Chen, Courey, A. J., BioTechniques 26, 814 (1999); D. Qi, K. B. Scholthof, J Virol Methods 149, 85 (April 2008); hereby incorporated by reference). The insert was positioned between the first and the fifth amino acids of the wild-type pVIII, replacing wildtype M13KE residues 2-4 (Ala-Glu-Gly-Asp-Asp (SEQ ID NO:51) to Ala-(Insert)-Asp). All primers were ordered from IDT DNA technologies (Coralville, Iowa). To allow for re-circularization of the vector following the PCR, a PstI restriction site was created upstream of the insert location using a QuikChange® Site-Directed Mutagenesis Kit by changing position 1372 of M13KE vector from T to A (CTGCÁG (SEQ ID NO:52)), as previously described (V. A. Petrenko, G. P. Smith, X. Gong, T. Quinn, Protein Eng 9, 797 (September 1996); hereby incorporated by reference). The resulting DNA was verified by picking blue plaques resulting from phage transformation, isolating the DNA using common biological methods (J. Sambrook, D. W. Russell, Molecular Cloning: A Laboratory Manual (CSHL Press, ed. 3rd, 2001)), and sequencing at the University of California, Berkeley DNA sequencing facility (Berkeley, Calif.). For the inverse PCR reaction, the forward primer was designed to include a PstI restriction site, an insert sequence, and a segment complimentary to the gVIII 3′-5′ strand. The reverse primer designed to make the M13 plasmid linear, also included the PstI restriction site and was fully complimentary to the gVIII 5′-3′ region. To incorporate the gene sequences, polymerase chain reaction (PCR) was performed using Phusion™ High-Fidelity DNA Polymerase, the two primers, and a PstI mutated M13 vector as the template. The obtained product was purified on an agarose gel, eluted with spin column purification, digested with PstI enzyme, and re-circularized with an overnight ligation at 16° C. with T4 DNA Ligase (New England Biolabs, Ipswich, Mass.). The ligated DNA vector was transformed into XL 10-Gold® Ultracompetent bacteria cells, and the amplified plasmid verified via DNA sequencing.

Partial Library Cloning Method: All the libraries were cloned into the M13 vector following the above scheme. For the partial libraries the primers were designed to constrain a region of interest (i.e. RGD), and to allow degeneracy within the flanking codons (i.e. XXXRGDXX). As in previous phage libraries, a 32 codon degeneracy was used (X═NNK, N=A/T/C/G, K=G/T) to reduce the bias among presented amino acids, and eliminate two of the three potential stop codons (V. A. Petrenko, G. P. Smith, X. Gong, T. Quinn, Protein Eng 9, 797 (September 1996); G. P. Smith, V. A. Petrenko, Chemical Reviews 97, 391 (March-April 1997); hereby incorporated by reference).

TABLE 1
Primer sequences for phages and phage libraries

| Library | Name Sequence | Primer^{§} |
|---|---|---|
| Wild-type | AEGDDP | |
| | (SEQ ID NO: 53) | |
| Unconstrained | AX*(X)₇DP . . . | 5′ATATATCTGCAGNK (NNK)₇ |
| | (SEQ ID NO: 54) | GATCCCGCAAAAGCGG |
| | | CCTTTAATCCC3′ (SEQ ID NO: 67) |
| RGD | AX*XX [symbol] XXDP | 5′ . . . CTGCAGNK (NNK)₂ [symbol] (NNK)₂ . . . 3′ |
| | (SEQ ID NO: 55) | (SEQ ID NO: 68) |
| WHWQ | A [symbol] DP | 5′ . . . CTGCAG [symbol] [symbol] [symbol] |
| | (SEQ ID NO: 56) | [symbol] . . . 3′ (SEQ ID NO: 69) |
| WHWQ-2X | AX* [symbol] X [symbol] DP | 5′ . . . CTGCAG NK [symbol] NNK [symbol] |
| | (SEQ ID NO: 57) | [symbol] . . . 3′ (SEQ ID NO: 70) |
| WHWQ-3X | AX* [symbol] XX [symbol] DP | 5′ . . . CTGCAG NK [symbol] (NNK)₂ |
| | (SEQ ID NO: 58) | [symbol] . . . 3′ (SEQ ID NO: 71) |
| WHWQ-4X | AX*X [symbol] XXDP | 5′ . . . CTGCAG NK NNK [symbol] |
| | (SEQ ID NO: 59) | (NNK)₂ . . . 3′ (SEQ ID NO: 72) |
| | AX* [symbol] XXXDP | 5′ . . . CTGCAG NK [symbol] (NNK)₃ . . . 3′ |
| | (SEQ ID NO: 60) | (SEQ ID NO: 73) |
| 1E** | AX*XX [symbol] XXXXDP | 5′ . . . CTGCAG NK (NNK)₂ [symbol] (NNK)₄ . . . 3′ |
| | (SEQ ID NO: 61) | (SEQ ID NO: 74) |
| 2E | AX*XX [symbol] XXXDP | 5′ . . . CTGCAG NK (NNK)₂ [symbol] (NNK)₃ . . . 3′ |
| | (SEQ ID NO: 62) | (SEQ ID NO: 75) |
| 3E | AX*X [symbol] XXXDP | 5′ . . . CTGCAG NK NNK [symbol] (NNK)₃ . . . 3′ |
| | (SEQ ID NO: 63) | (SEQ ID NO: 76) |
| 4E | AX*X [symbol] XXDP | 5′ . . . CTGCAG NK NNK [symbol] ₂ (NNK)₂ . . . 3′ |
| | (SEQ ID NO: 4) | (SEQ ID NO: 77) |
| 5E | AX* [symbol] XXDP | 5′ . . . CTGCAG NK [symbol] (NNK)₂ . . . 3′ |
| | (SEQ ID NO: 65) | (SEQ ID NO: 78) |
| 6E | AX* [symbol] XDP | 5′ . . . CTGCAG NK [symbol] 3 NNK . . . 3′ |
| | (SEQ ID NO: 66) | (SEQ ID NO: 79) |

Xs refer to unconstrained residues, Bold to constrained, and Italics to the insert portion
*X refers to residues coded for by a GNK codon (i.e. A, D, E, G, V)
**Glu libraries shown as representative, same pattern applied for Lys library (AAA & AAG codons), for His (CAT), and W (TGG) libraries only one type of codon was used
^{§}Full primer sequence shown only for the 1^{st} listed, for the rest only the PstI restriction site and the insert portion is included

For a fully unconstrained library, degeneracy was allowed at all of the 8 positions. See Table 1 for primer and corresponding library sequences. To reduce the advantage of wild-type or fast-amplifying phage, the cells were allowed to recover for only 30 minutes after transformation (K. A. Noren, C. J. Noren, Methods 23, 169 (February 2001); hereby incorporated by reference). To analyze the library sequence space the cells were plated in agarose top, and plaques formed after overnight incubation at 37° C., were then picked and sequenced.

Genetic Engineering for Multifunctional Phage. Using inverse PCR and partially constrained library methods described above (A. Merzlyak, S. Indrakanti, S. W. Lee, Nano Lett 9, 846 (2009); hereby incorporated by reference), we were able to display a variety of functional motifs on pVIII and pIII phage proteins. RGD and IKVAV (SEQ ID NO:1) are well known peptides utilized for regulating and promoting cell adhesion and differentiation of neural progenitor cells (A. Merzlyak, S. Indrakanti, S. W. Lee, Nano Lett 9, 846 (2009); K. Saha, E. F. Irwin, J. Kozhukh, D. V. Schaffer, K. E. Healy, J Biomed Mater Res A 81, 240 (2007); J. C. Schense, J. Bloch, P. Aebischer, J. A. Hubbell, Nat Biotechnol 18, 415 (2000); G. A. Silva et al., Science 303, 1352 (2004); all of which are hereby incorporated by reference). The above peptides along with their nonspecific controls RGE, and IQVAV (SEQ ID NO:1) were engineered on pVIII, for a dense signal displayed for tissue engineering applications. HPQ is a biotin-like motif found previously to bind streptavidin molecules. An HPQ conjugated phage can be used in a variety of biotechnological application to deliver streptavidin conjugated therapeutics to the target cells (A. Hajitou et al., Cell 125, 385 (2006); hereby incorporated by reference). HPQ was displayed in a linear form on pVIII as FSHPQNT (SEQ D NO:112) (Kd=125 μM) (J. J. Devlin, L. C. Panganiban, P. E. Devlin, Science 249, 404 (1990); P. C. Weber, M. W. Pantoliano, L. D. Thompson, Biochemistry 31, 9350 (1992); all of which are hereby incorporated by reference), and in a cys-constrained form C-HPQGPP-C (SEQ ID NO:80) (Kd=0.230 μM)(16) on pIII. Although the constrained HPQ motif has been found to have a much higher affinity of binding for streptavidin, there are only 5 copies of pIII protein per phage, and so the potential applications of delivery are different for the differently designed phage, depending on the necessary delivery of the therapeutic molecule. Furthermore to deliver therapeutic molecules to cells, multifunctional phage were designed with pVIII RGD and pIII HPQ and vice versa, along with the non-physiological controls, on wild-type or RGE phage (Table 2).

TABLE 2
Genetically Engineered Functionalized
p8 and p3 phage

| | | |
|---|---|---|
| Name | PVIII | PIII |

p8 and p3 Physiologically Relevant Sequences

| | | |
|---|---|---|
| p8-RGD | A [symbol] DP | |
| | (SEQ ID NO: 80) | |
| p8-RGE | A [symbol] DP | |
| | (SEQ ID NO: 81) | |
| p8-RGD | A [symbol] EDP | |
| scram | (SEQ ID NO: 82) | |
| p8-IKVAV | A [symbol] DP | |
| | (SEQ ID NO: 83) | |
| p8-IQVAV | A [symbol] DP | |
| | (SEQ ID NO: 84) | |
| p8-DGEA | A [symbol] DP | |
| | (SEQ ID NO: 85) | |
| p8-HPQ | AE [symbol] DP | |
| | (SEQ ID NO: 86) | |
| p3-RGD | | A- [symbol] SAE |
| | | (SEQ ID NO: 105) |
| p3-RGE | | A- [symbol] SAE |
| | | (SEQ ID NO: 106) |
| p3-HPQ | | A- [symbol] SAE |
| | | (SEQ ID NO: 107) |
| p8-HPQ/ | AE [symbol] DP | A- [symbol] SAE |
| p3-RGD | (SEQ ID NO: 87) | (SEQ ID NO: 108) |
| p8-HPQ/ | AE [symbol] DP | A- [symbol] SAE |
| p3-RGE | (SEQ ID NO: 88) | (SEQ ID NO: 109) |
| p8-RGD/ | AD [symbol] DP | A- [symbol] SAE |
| p3 HPQ | (SEQ ID NO: 89) | (SEQ ID NO: 110) |
| p8-RGE/ | A [symbol] DP | A- [symbol] AE |
| p3 HPQ | (SEQ ID NO: 90) | (SEQ ID NO: 111) |

p8-Libraries

| | |
|---|---|
| p8-WHWQ | A [symbol] DP |
| | (SEQ ID NO: 91) |
| | A [symbol] DP |
| | (SEQ ID NO: 92) |
| | A [symbol] DP |
| | (SEQ ID NO: 93) |
| p8-1H | A [symbol] DP |
| | (SEQ ID NO: 94) |
| p8-2H | A [symbol] DP |
| | (SEQ ID NO: 95) |
| p8-3H | A [symbol] DP |
| | (SEQ ID NO: 96) |
| p8-1K | A [symbol] DP |
| | (SEQ ID NO: 97) |
| p8-1W | A [symbol] DP |
| | (SEQ ID NO: 98) |
| p8-2W | A [symbol] DP |
| | (SEQ ID NO: 99) |
| p8-3W | A [symbol] DP |
| | (SEQ ID NO: 100) |
| p8-1E | A [symbol] DP |
| | (SEQ ID NO: 101) |
| p8-2E | A [symbol] DP |
| | (SEQ ID NO: 102) |
| p8-3E | A [symbol] DP |
| | (SEQ ID NO: 103) |
| p8-4E | A [symbol] DP |
| | (SEQ ID NO: 104) |

Peptide inserts are shown in italic and bold, functional sequences are also underlined.

IKVAV- (SEQ ID NO:1) and RGD-phage engineered as described above to display peptides at the N-termini of the pVIII major coat proteins, and the wild-type M13 phage were amplified to 12-liter volumes via E. coli infection (FIG. 4A). The phage were then purified by PEG precipitation. Prior to proceeding with cell culture work, the phage were filtered through a 0.45 μm syringe filter to further remove any remaining bacterial particles. The final concentration of amplified phage was on the order of 5×10 phage/mL.

Construction and characterization of phage-based tissue scaffold structures. One-dimensional phage-fibers were fabricated using electrospinning and conventional wet-spinning (Lee, S.-W. and A. M. Belcher, Virus-Based Fabrication of Micro-and Nanofibers Using Electrospinning. Nano Letters, 2004. 4(3): p. 387-390; hereby incorporated by reference). Long-range ordered smectic liquid crystalline films were fabricated through meniscus flowing force-assisted cast film processes (Lee, S. W., et al., Ordering of quantum dots using genetically engineered viruses. Science, 2002, 296(5569): p. 892-895; Lee, S.-W., S. K. Lee, and A. M. Belcher, Virus-based alignment of inorganic, organic, and biological nanosized materials. Advanced Materials (Weinheim, Germany), 2003, 15(9): p. 689-692; hereby incorporated by reference). Using these well-established techniques, we constructed one-dimensional fibers and two-dimensional cast films as described below.

Construction of liquid crystalline solutions using genetically engineered phage. We constructed liquid crystalline solutions using pVIII-engineered phage (FIG. 4A). ˜100 mg of phage pellet was suspended in 1 ml of TBS buffer to prepare a smectic phase liquid crystalline suspension. The resulting suspension was diluted to prepare the isotropic, nematic, cholesteric, and smectic phases of the liquid crystalline structures. Polarized optical micrographs were used to determine the phases [i.e., nematic (10-25 mg/ml), cholesteric (40-75 mg/ml), and smectic (90-120 mg/ml, FIG. 4B)], and the structures formed by all the engineered phage were very similar. After critical point drying the samples in the smectic phase spontaneously aligned and could be visualized as individual phage nanofibers (FIG. 4C). The self-aligned genetically engineered phage-based materials were able to display the signaling motifs with directionality and, therefore could guide the neuronal cells through directional contact guidance.

Fabrication of one-dimensional fibers. We fabricated one-dimensional fibers using mild spinning conditions by spinning the liquid crystalline suspensions (20-100 mg/ml) into an ethanol fixative (70% aqueous ethanol). FIG. 4D shows the viral fibers with IKVAV-engineered phage. Scanning electron microscopy images (FIGS. 4E and F) show that the long axes of the phage are parallel to the long axis of the viral fibers.

Fabrication of liquid crystalline films. We fabricated two-dimensional ordered liquid crystalline phage films (FIG. 4G). 1.5 mg/ml of phage suspension was cast dried on the surface of a vinyl film substrate (Thermanox™ cell culture cover slips, Nalge Nunc International, Rochester, N.Y.) in a dessicator for one week. Examination by brightfield and phase contrast microscopy revealed striped band patterns in the dried sample, indicating that the phage long axes were aligned perpendicularly to the bands in a smectic liquid crystalline alignment (FIGS. 4H and I).

### EXAMPLE 8

### Self-Templating Chiral Supramolecular Structures

Self-templating is a commonly observed material design theme in nature, whereby preexisting structures determine the organization of subsequent structures. Here, we developed novel bio-inspired “self-templating” hierarchical structures using a chiral colloidal particle, M13 phage. By controlling the interplay between competing interfacial forces and liquid crystalline phase transitions of phage solutions, we created novel chiral structures with controllable long range order on the centimeter scale. The resulting structures were used as selective optical reflectors/filters and grid prisms and exhibited structural colors commensurate with their structures. Using genetically engineered M13 phages, we also developed bio-inspired functional biomaterials for soft and hard tissue regeneration. Our phage-based model system provides insight into understanding the complexities of the hierarchical self-assembly of biomolecules in biological process.

The design of hierarchical structures ordered over lengths ranging from the nano- to macroscale is of great interest in many science and engineering fields including chemistry, physics, biology, material science, and electric engineering. Various bottom-up self-assembly processes have been used to create higher order structures from inorganic and organic nanoparticles, nanowires, and nanosheets (1-4). The resulting structures have exhibited exquisite electrical and optical properties (5, 6) and performed biomedical functions such as drug delivery or tissue regeneration (7-9). Recently, top-down self-assembly processes have created complex structures that can be used to develop sophisticated microelectronics and photonic devices (10-12). Microfabricated nanocolumn structures can be induced to form helical structures and undergo biomimetic actuating motions through the control of interfacial forces (13, 14). Although these examples have demonstrated the potential of using self-assembly processes to develop novel functional materials, the functional and structural complexity of manmade materials does not compare to that of natural systems (e.g. diatoms, seashells, bones) (15-17).

In nature, helical molecules such as DNA, collagen, chitin, cellulose, and many other biological macromolecules play a critical role in life processes and in the formation of structures during the morphogenesis of cells, tissues, and organs (19, 20). In addition, these chiral building block materials are used to create diverse hierarchical structures that are made by selftemplating, in which pre-existing structures in given microenvironments determine the organization of subsequent structures. Synergic interactions between neighboring molecules result in the formation of materials with varied qualities. For example, type I collagen can selfassemble into various hierarchical structures with tunable properties: orthogonally aligned collagen fibers are found in optically transparent corneal tissues (18), whereas the twisted nematic phase of collagen fiber bundles is found in the blue-colored soft tissues of many avian and mammalian skins (19, 20). Diverse, hierarchically organized collagen fibers interact with surrounding proteins and molecules to create tissues that range from soft cartilage to hard, mineralized teeth and bones (15). However, due to limitations in modifying biological building blocks and limited knowledge about their self-assembly, the study of the relationship between hierarchical structures and their diverse functions is challenging. Here, we report a novel approach of using M13 bacteriophage (phage), a monodisperse chiral mesogen, to construct various hierarchical structures. We created self-templating supramolecular structures by controlling the interfacial forces and liquid crystalline phase transitions of phage suspensions deposited on a substrate from the suspension's meniscus. In the deposition process, the helicity of the M13 phage played a critical role in controlling the resulting self-templated structures. The periodic structures exhibited tunable optical properties with transparent to full range color characteristics. In addition, by genetically engineering the phage, we could impart specific biological functions to the resulting ordered structures to guide the growth of soft and hard tissues. Our approach can be expanded to the supramolecular assembly of many other chiral molecules and can be used to construct sophisticated functional chiral structures for the development of optical and photonic materials and tissue regenerating materials. In addition, our approach provides a means for better understanding the self-assembly of chiral molecules and gives insight into hierarchical chiral structure-function relationships in nature.

M13 phage is a filamentous bacterial virus (21). It is covered along its length with 2700 alpha helical protein subunits (pVIII) that exist in a right-handed helical arrangement with fivefold symmetry (FIG. 14A) (22, 23). Because of their well-defined shape and the ease by which they can be prepared in large quantities, M13 and other rod-shaped viral particles have been used as model systems for studying complex fluids. They organize into liquid crystalline phases such as nematic, cholesteric (twisted nematic or chiral nematic), and smectic phases (24-27). These phases are dependent on the concentration of the viral suspension, ionic concentration, and the application of external fields (e.g., magnetic or electric fields and meniscus forces) (28). By exploiting the well-defined structure and genetic versatility of M13, one can design several novel functional nanostructures. For example, genetically engineered M13 phages that have been subjected to a directed evolution process have been used to develop semiconductor and energy storage materials (28-31).

We created diverse self-templating supramolecular structures by modulating the interfacial forces and liquid crystal phase transitions of phage solutions along a meniscus (FIG. 14). First, a substrate was vertically immersed in an M13 phage suspension and pulled up at a controlled speed. At the air/liquid/solid interface, evaporation proceeds fastest near the meniscus contact line (FIG. 14B) to induce various liquid crystalline phase transitions (from isotropic to nematic or smectic phases) of the viral colloidal suspension on the substrate. These transitions were found to depend on the initial phage concentrations. As the concentration of the phage suspension increased, the viscosity also increased, and the suspension stuck to the substrate at the meniscus line where the meniscus was pinned by deposited phages/phage bundles and stretched by the rising substrate. When the pinning force of the sticky suspension reached a critical point and could no longer overcome the surface tension generated by pulling the substrate, the meniscus broke and then slipped. The interplay between these competing interfacial forces and the accompanied liquid crystalline phase transitions resulted in the formation of diverse hierarchically ordered supramolecular structures. Because this interplay continuously occurred within a range of tens of micrometers as the substrate was pulled up in controlled manner, long range ordered periodic supramolecular structures could be created (FIG. 14C). Using this process to deposit phage films, we identified three novel and distinctive self-templated structures: alternating nematic stripe, cholesteric helix ribbon, and chiral smectic O* nanofilament structures. We analyzed the morphologies of the resulting self-assembled microstructures using polarized optical microscopy (POM), scanning electron microscopy (SEM), atomic force microscopy (AFM), light diffraction, and computational modeling.

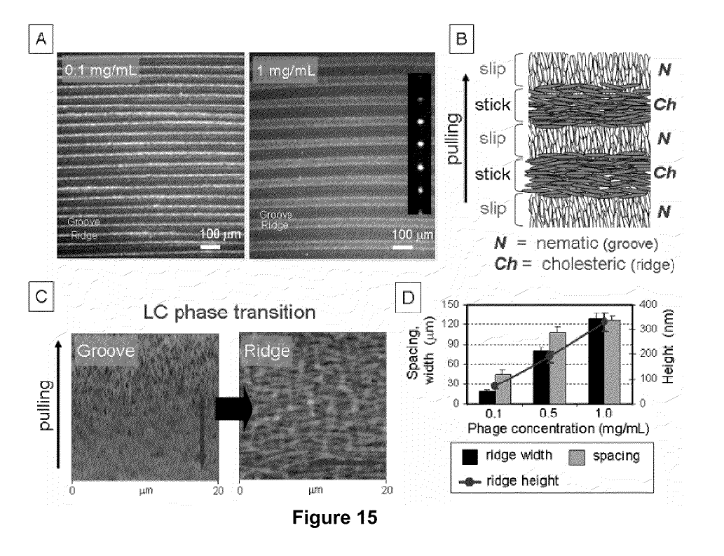

We fabricated the alternating nematic striped pattern at a low phage concentration (0.05-2.0 mg/mL), with stripes forming perpendicular to the pulling direction of the substrate (FIG. 15A). AFM microstructure analysis revealed that these stripe patterns were composed of alternating ridge and groove structures formed by the selective and periodic deposition of phages. Depending on the phage concentration, different types of nematic liquid crystalline phase transitions were observed on the ridges. During evaporation, the phages first formed liquid crystalline phases at the meniscus and then aggregated to form bundled fibers that behaved as supramolecular mesogens and organized into hierarchically ordered supramolecular structures. In a lower concentration range (0.05-0.1 mg/mL), nematically oriented phage bundles were observed on both the ridge and groove structures. The average width of the bundled fibers in the ridges was greater than that in the grooves (˜270 nm vs.˜75 nm, respectively) due to the longer deposition time during the pinning event. At a higher concentration range (0.2-2.0 mg/mL), we observed alternating nematic-cholesteric (twisted nematic) phase structures in periodic ridge and groove patterns as shown in the schematic illustration (FIG. 15B). AFM analysis showed that the nematic phage bundle fibers were oriented parallel to the pulling direction of the substrate in the grooves (FIG. 15C left), whereas the phage bundle fibers were aligned at 90° to the pulling direction at the ridges (FIG. 15C right). When the meniscus was pinned to form the ridge, liquid crystalline phage bundles were induced to stack in layers with a tilted director axis. Thus, the evaporation of the solvent resulted in the accumulation of the twisted nematic layers perpendicular to the pulling direction. However, when the meniscus receded and slipped, the nematic phage bundles were forced to align parallel to the pulling direction. These periodic alternating nematic and cholesteric phase structures exhibited regular spacing and were observed over centimeter scales throughout the samples. They exhibited clear diffraction patterns reflecting their regular spacing (FIG. 15A, inset) and characteristic colors depending on their thickness and bundled fiber diameters. These self-templating periodic structures were tunable by varying the concentration of the phage suspension, the pulling speed, and the surface chemistry, all of which affect the meniscus forces (F_{surface tension}=pgh: p: density of phage suspension; g: gravity; h: height) and friction forces (F_{friction}=−kx, k=friction constant; x=movement). Increasing the phage concentrations caused the widths, heights, and interspacing of the ridges to increase; whereas increasing the pulling speed (0.5-2.0 mm/h) caused ridge widths, heights and interspacing to decrease (FIG. 15D). Higher initial phage concentrations (still in the isotropic phase) and slower pulling speeds allowed for greater phage deposition at the meniscus line due to the increased duration of pinning, resulting in greater ridge width and height.

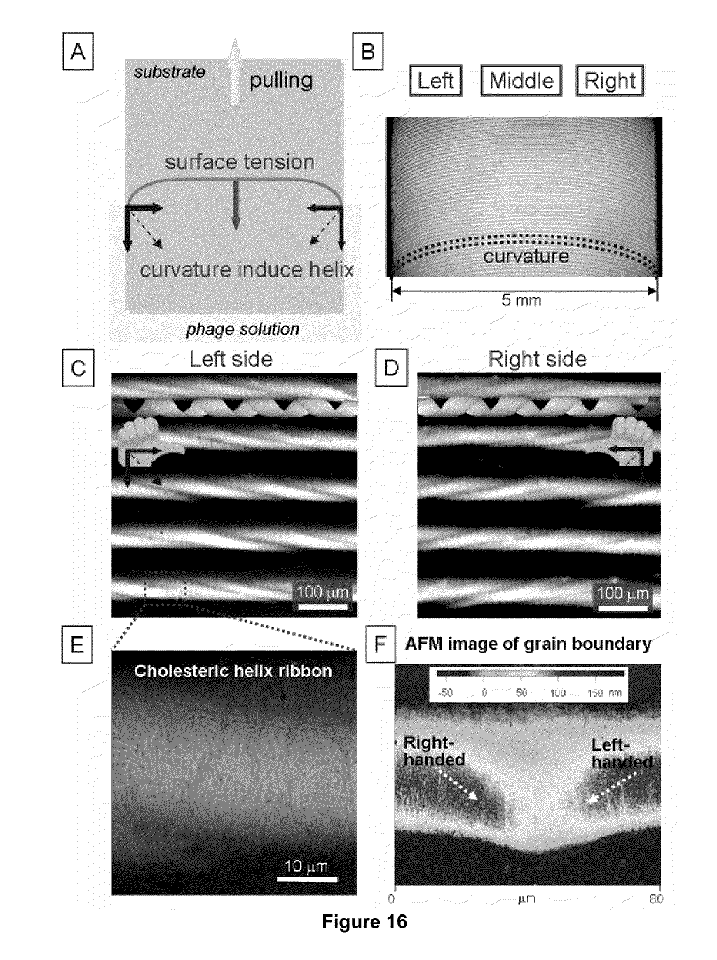

We also constructed self-templating cholesteric helix ribbon structures by controlling the geometry of the meniscus (FIGS. 16A and B). At phage concentrations greater than 0.25 mg/mL, ionic concentrations lower than 200 mM, and a pulling speed near 1 mm/h, a significant curvature was induced at the outer edges of the meniscus. Under these conditions, we observed that the outer edges of the meniscus had three-dimensional (3D) curved shapes and mirrored each other (shown in FIG. 16A), whereas the middle area of the meniscus was two dimensional (2D) and flat (FIG. 16A). Faster evaporation occurred at the outer edges due to the increased surface areas at the curves, resulting in faster phase transitions of the phage suspension on the substrate than those occurring at the meniscus line in the middle area. At the outer edges, the force caused by the surface tension acted downward and inward (solid arrows in FIG. 16A), exerting a driving force that twisted the deposited structures (dotted arrows in FIG. 16A). The twisted helical structures initiated at the outer edges significantly influenced the deposition of the following supramolecular structures toward an inward direction along the meniscus line. The twisted ridge patterns exhibited left- and right-handed helical ribbon structures that met at the center, reflecting the mirrored images of the meniscus edges: the left side of the pattern exhibited a right-handed twisted ribbon structure (FIG. 16C), whereas the right side exhibited a left-handed twisted ribbon structure (FIG. 16D). Interestingly, the predeposited helical ridges controlled the periodicity of the following helical structures. Through AFM analysis, we confirmed that these left- and right-handed helical ribbons possessed a twisted plywood structure, corresponding to the oblique cross-section of the cholesteric phase of the phage particles (dashed arch lines in FIG. 16E). Despite the opposite handedness of the twisting, the surfaces of the ridges on both sides of the meniscus displayed the same handedness of the cholesteric phase. At the middle where these two cholesteric helix ribbons met, we observed a grain boundary with constrained structures (FIG. 16F). By exploiting the 3D curvature at the outer edges of the menisci, we could construct controlled left- and right-handed macroscopic cholesteric helix ribbons using right-handed phage-based cholesteric structures.

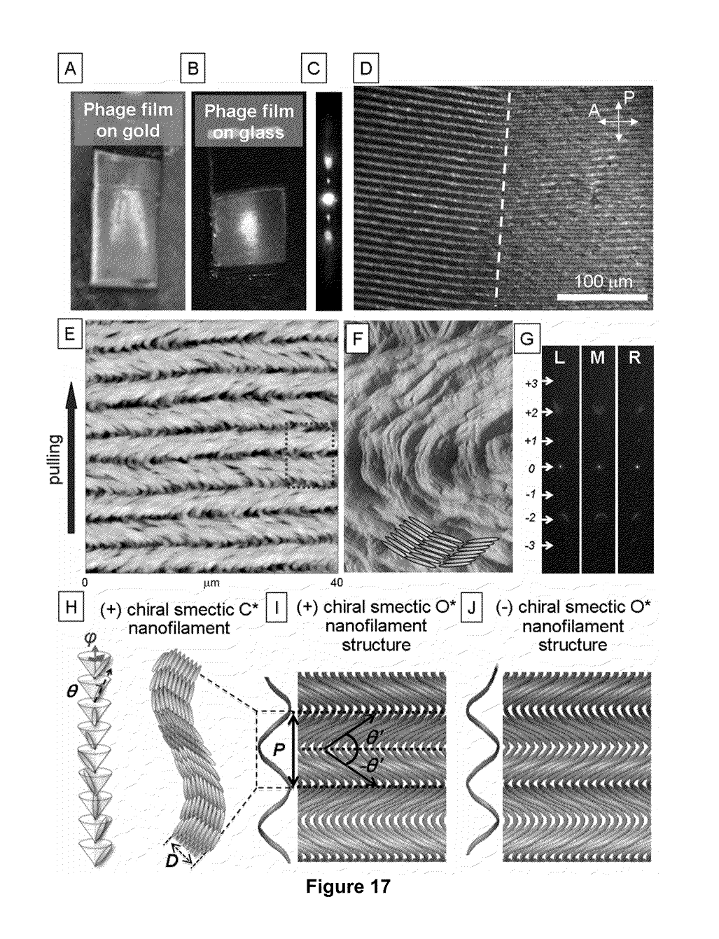

At higher concentrations (4-6 mg/mL) iridescent prism-like optical films were created (FIGS. 17A and B). When the phage film on a gold-coated substrate was spot illuminated with a white light (˜1 mm diameter), the film exhibited multiple ordered V-shaped iridescent diffraction patterns (FIG. 17A). Similar iridescent diffraction patterns of reflected and transmitted light were observed with films grown on glass substrates (FIGS. 17B and C). POM images revealed that the iridescent diffraction originated from grid-like periodic patterns with 9.1 μm pitch spacing (FIG. 17D). Similar to the cholesteric helix ribbon structures (FIG. 17F), these films also exhibited a grain boundary in the middle area of the film with opposite polarized light responses to each other across the boundary (dashed line in FIG. 17D). AFM microstructure analysis revealed that the grid-like periodic patterns were composed of stacked chiral smectic C phage fibers constituting smectic O phase zig-zag structures (FIGS. 17E and F). Both left- and right-sides of the films possessed the same “ramen noodle-like” morphology on the centimeter scale. When He—Ne laser light (632.8 nm) was used to illuminate either side of the films, the films exhibited different distinctive diffraction patterns depending on the areas illuminated (left, middle, and right areas in FIG. 17G). We confirmed that the diffraction pattern resulted from the combined effect of the periodicity of the zig-zag pattern with regular spacing and the angles of the chiral smectic C fiber bundles constituting zig-zag structures. The diffraction pattern exhibited a very weak first-order diffraction spot, correlating to 9.1 μm zig-zag pattern spacing. A strong second-order spot correlated to the 4.6 μm half pitch of those patterns. We named the resulting structure “chiral smectic O* nanofilament phase”, which was composed of chiral smectic O* phase structures based on chiral smectic C* bundle phage nanofilaments. Based on our microscopy and diffraction analysis results, we were able to construct a mathematical model of the smectic O* nanofilament phase structure and reproduce the observed diffraction patterns through computation. First, the smectic C bundle was taken as the basic unit of structure of the filament phase, displaying smectic tilt angle (θ) and azimuthal rotation (φ) (FIG. 17H). We then applied a hypothetical herringbone-like model structure having the following parameters: the diameter (D) of the phage bundle unit structure (900 nm), the tilt angle (θ′) of the smectic nanofilament phase angle (±62°, and the periodic pitch (P; 9.1 μm) (FIG. 17I). When the bundles were stacked together, their helicity was still retained in the resulting bulk structure, which induced the formation of the zig-zag chiral smectic O* phase with helical (right handed (+) or left-handed (−)) rotation (FIGS. 17I and J). Due to the curved geometry of the meniscus, the deposited structures possessed mirror image symmetry and applied mirrored twisting forces from either edge of the meniscus. The characteristic tilted symmetric diffraction patterns (FIG. 17G) observed from the left- and right-side of films could be attributed to the mirrored curvature of the films and the left and right-handedness of the filament phase (FIGS. 17I and J). These novel structures exemplify the higher order morphologies that can be induced during the self-templating phage film growth process.

In an effort to mimic various helical structures in nature, we created functional phage films with tunable properties for optical materials and tissue regenerating materials (FIG. 18). By changing the pulling speeds between 20-80 μm/min, we constructed a full range of structural color band patterns composed of the smectic O* nanofilament phase (FIG. 10A). The colors of the patterns changed depending on the angle of incident light. Reflectance measurements showed that the characteristic optical spectra correlated to the corresponding colors (FIG. 18B). We believe that the different colors were the collective result of the different phage bundle diameters and phage film thicknesses. We also fabricated optical reflectors and filters composed of alternating smectic O* nanofilaments and nematically aligned phage fibers structures, in which horizontally and vertically oriented macroscopic structures were created (FIG. 18C-E) by modulating the pulling speeds. These virus-based optical reflectors and filters selectively scattered visible light or allowed light to pass through, depending on the direction of incident light.

Inspired by the fact that hierarchically organized nanofilament structures play a critical role in controlling the morphogenesis and function of desired tissues and organs, we constructed biomimetic tissue scaffolds using self-templating phage structures. We genetically modified the phage to display an RGD-integrin binding peptide or an osteopontin-like highly negative charged peptide motif (tetra-glutamate EEEE (SEQ ID NO:105)) to mimic natural soft and hard tissue templates. When MC-3T3 preosteoblast cells were cultured on top of the directionally organized RGD-phage films, they recognized the aligned microstructures and biochemical cues (RGD motifs) of the films and grew along the direction of the viral film microstructures (FIG. 18F-I). When we treated phage films composed of both RGD-phage and EEEE (SEQ ID NO:105)-phage (1:1) with Ca²⁺ and PO₄³⁻ solutions, the films induced biomineralization of bone (hydroxyapatite), resulting in a hierarchically organized dentin-like organic-inorganic composite structures (FIG. 18J). We found that the stiffness (Young's modulus) of the phage film was significantly increased (˜18 times) after biomineralization (FIG. 18K). We were, therefore, able to create materials with diverse optical, photonic, and biomimetic tissue regenerating functions using hierarchical microstructures of the engineered phage.

Theoretical modeling of the continuum elastic behavior of chiral bundles has predicted that rod-like molecules with handedness or lack of mirror symmetry prefer to induce mutual torque between neighboring molecules, resulting in twisted helical arrangements (32). Due to the incompatibility between ordering and twisting during the crystallization of the chiral molecules in a confined space, it has been known that various chiral or achiral materials form higher ordered chiral structures including cholesteric (25), chiral smectic C* (27), or helical nanofflament phases (33). Using the M13 system, we demonstrated multiple new findings related to biological structures. First, our self-assembled microstructures exhibited a novel selftemplating ability, in which pre-deposited structures controlled the morphology of subsequently deposited structures. Second, we showed that both left-handed and right-handed macroscopic helical structures could be formed regardless of the intrinsic helicity of the building block molecules. Third, the resulting self-templating liquid crystalline structures exhibited controllable long range order on the centimeter scale, whereas fewer crystalline ordered structures were observed on shorter scales. Finally, genetic modification of the viral particles greatly expanded the functions of the resulting hierarchical structures. The selective display of peptides that bind to synergistic molecules on the viral structures could have several significant applications in tissue regeneration and bone biomineralization.

In summary, we developed novel self-templating helical supramolecular structures using monodisperse viral colloidal nanoparticles. Self-templating was induced mainly by the helical structure of the phage bundles and the interfacial forces in the meniscus areas. The interplay between the competing interfacial forces and liquid crystal transitions played a critical role in producing diverse left- and right-handed helix supramolecular structures. Using various microscopy techniques, optical characterization, and mathematical modeling, we elucidated the structure-function relationship of the supramolecular helix structures. Our phage-based study may provide insight into understanding the complex self-assembly of biomolecules and the selftemplating process that is commonly observed in living systems. The resulting helical structures could be useful in fabricating advanced optical and photonic materials and tissue regenerating materials.

### REFERENCES

1. S. I. Stupp et al., Science 276, 384 (1997).
2. A. P. Alivisatos, Science 271, 933 (1996).
3. J. D. Holmes, K. P. Johnston, R. C. Doty, B. A. Korgel, Science 287, 1471 (2000).
4. J. S. Son et al., Angew. Chem.-Int. Edit. 48, 6861 (2009).
5. C. A. Mirkin, R. L. Letsinger, R. C. Mucic, J. J. Storhoff, Nature 382, 607 (1996).
6. D. H. Gracias, J. Tien, T. L. Breen, C. Hsu, G. M. Whitesides, Science 289, 1170 (2000).
7. E. P. Holowka, V. Z. Sun, D. T. Kamei, T. J. Deming, Nat. Mater. 6, 52 (2007).
8. G. A. Silva et al., Science 303, 1352 (2004).
9. R. G. Ellis-Behnke et al., Proc. Natl. Acad. Sci. U.S.A. 103, 5054 (2006).
10. S. Park et al., Science 323, 1030 (2009).
11. S. O. Kim et al., Nature 424, 411 (2003).
12. I. Bita et al., Science 321, 939 (2008).
13. A. Sidorenko, T. Krupenkin, A. Taylor, P. Fratzl, J. Aizenberg, Science 315, 487 (2007).
14. B. Pokroy, S. H. Kang, L. Mahadevan, J. Aizenberg, Science 323, 237 (2009).
15. S. Weiner, W. Traub, H. D. Wagner, J Struct Biol 126, 241 (1999).
16. N. Kroger, S. Lorenz, E. Brunner, M. Sumper, Science 298, 584 (2002).
17. A. M. Belcher et al., Nature 381, 56 (1996).
18. D. F. Holmes et al., Proc. Natl. Acad. Sci. U.S.A. 98, 7307 (2001).
19. R. O. Prum, R. Tones, J Exp Biol 206, 2409 (2003).
20. R. O. Prum, R. Tones, J Exp Biol 207, 2157 (2004).
21. B. Hohn, H. Lechner, D. A. Marvin, J. Mol. Biol. 56, 143 (1971).
22. D. A. Marvin, L. C. Welsh, M. F. Symmons, W. R. Scott, S. K. Straus, J Mol Riot 355, 294 (2006).
23. S. K. Straus, W. R. P. Scott, D. A. Marvin, Eur. Biophys. J. Biophys. Lett. 37, 1077 (2008).
24. Z. Dogic, S. Fraden, Phys. Rev. Lett. 78, 2417 (1997).
25. Z. Dogic, S. Fraden, Langmuir 16, 7820 (2000).
26. K. R. Purdy et al., Phys. Rev. E 67 (2003).
27. S. W. Lee, B. M. Wood, A. M. Belcher, Langmuir 19, 1592 (Mar. 4, 2003).
28. S. W. Lee, C. B. Mao, C. E. Flynn, A. M. Belcher, Science 296, 892 (2002).
29. Y. J. Lee et al., Science 324, 1051 (2009).
30. C. B. Mao et al., Science 303, 213 (2004).
31. K. T. Nam et al., Science 312, 885 (2006).
32. G. M. Grason, Phys. Rev. E 79 (2009).
33. L. E. Hough et al., Science 325, 456 (2009).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising a modified bacteriophage capable of guiding cell growth and polarization via signaling peptides and directionally aligned structures.

2. The composition of claim 1, wherein the modified bacteriophage is a modified M13 bacteriophage comprising one or more recombinant phage coat protein comprising a signal peptide capable of promoting or causing a desired biological effect.

3. The composition of claim 2, wherein the coat protein is pIII, pVIII, or pIX.

4. The composition of claim 1, wherein the signal peptide is 3-8 amino acids long.

5. The composition of claim 4, wherein the signal peptide comprises the amino acid sequence of RGD, HPQ, HAV, EQS, or any amino acid sequence described by SEQ ID NO:1-42.

6. The composition of claim 5, wherein the signal peptide comprises IKVAV (SEQ ID NO:1), GRGD (SEQ ID NO:3), DGEA (SEQ ID NO:2), YIGSR (SEQ ID NO:4), RGD, or HPQ.

7. The composition of claim 1 comprising a phage matrix comprising a plurality of the modified bacteriophages, wherein the modified bacteriophages a directionally aligned to each other.

8. The composition of claim 6, wherein the phage matrix is a 2-D phage film, or any other 2-D structure, or a 3-D structure.

9. The composition of claim 8, wherein the phage matrix comprises a self-aligned structure.

10. The composition of claim 9, wherein the self-aligned structure comprises an alternating nematic-cholesteric structure.

11. The composition of claim 9, wherein the self-aligned structure comprises a cholesteric helix ribbon structure.

12. The composition of claim 9, wherein the self-aligned structure comprises a chiral smectic O* nanofilament structure.

13. The composition of claim 8 comprising a tissue matrix comprising viable cells in contact with the phage matrix.

14. The composition of claim 13, wherein the viable cells are osteoblasts, chondroblasts, hepatocytes, enterocytes, urothelials, neural cells, or fibroblasts.

15. The composition of claim 1, wherein the desired biological effect is transport, promote cell interaction, or regulate a cellular behavior.

16. The composition of claim 1, wherein the signal peptide is an adhesion peptide sequence, enzymatic substrate peptide sequence, or heparin binding peptide sequence.

17. A recombinant or purified nucleic acid encoding a modified M13 bacteriophage comprising one or more recombinant phage coat protein comprising a signal peptide capable of promoting or causing a desired biological effect.

18. A method of constructing the composition of claim 4 comprising: (a) providing a plurality of modified M13 bacteriophage comprising one or more recombinant phage coat protein comprising a signal peptide capable of promoting or causing a desired biological effect, and (b) allowing for self-alignment or applying an external force to align the plurality of recombinant M13 phages.

19. The method of claim 18, further comprising: (c) adding one or more viable cells of interest to the phage matrix to form a tissue matrix

20. The method of claim 19, further comprising: (d) culturing the tissue matrix for a sufficient period of time such that the viable cells grow on the phage matrix.

21. The method of claim 20, further comprising: (e) implanting the tissue matrix into a subject.

22. The method of claim 21, wherein the viable cells are originally obtained from the subject.

23. The method of claim 22, wherein the viable cells are osteoblasts, chondroblasts, hepatocytes, enterocytes, urothelials, neural cells, or fibroblasts.
